(19) United States
(12) Patent Application Publication
(10) Pub. No.: US 2007/0123491 A1
(43) Pub. Date: May 31, 2007

(54) Podophyllotoxin derivatives as igf-1r inhibitors

(75) Inventors: Magnus Axelson, Hasselby (SE); Olle Larsson, Taby (SE)

Correspondence Address: OBLON, SPIVAK, MCCLELLAND, MAIER & NEUSTADT, P.C., 1940 DUKE STREET, ALEXANDRIA, VA, 22314, US

(73) Assignee: BIOVITRUM AB, Stockholm (SE)

(21) Appl. No.: 10/554,399

(22) PCT Filed: Apr. 15, 2004
(86) PCT No.: PCT/SE04/00590

§ 371 Date: Feb. 2, 2007

Related U.S. Application Data
(60) Provisional application No. 60/468,054, filed on May 6, 2003.

(30) Foreign Application Priority Data
Apr. 24, 2003 (SE) 0301202-8

(51) Int. Cl. A61K 31/695; A61K 31/47; A61K 31/444; A61K 31/44; A61K 31/382; A61K 31/353; A61K 31/215; A61K 31/165

(52) U.S. Cl. 514/63; 514/256; 514/357; 514/312; 514/456; 514/332; 514/432; 514/114; 514/529; 514/617; 514/620; 514/452; 514/463

(57) ABSTRACT

The invention refers to new compounds, e.g. podophyllotoxin derivatives, as well as to the use thereof and of known compounds as specific inhibitors of the insulin-like growth factor-1 receptor (IGF-1R). Said compounds can be used for treatment of IGF-1/IGF-1R dependent diseases, such as cancer, psoriasis, arteriosclerosis, certain endocrine and metabolic disorders etc.

The present invention refers to new compounds as well as to the use thereof and of known compounds as specific inhibitors of the insulin-like growth factor-1 receptor. Said compounds can be used for treatment of IGF-1/IGF-1R dependent diseases, such as cancer, psoriasis, arteriosclerosis, certain endocrine and metabolic disorders etc.

### BACKGROUND OF THE INVENTION

The insulin-like growth factor-1 (IGF-1) and its receptor (IGF-1R) play important roles for the development of many diseases, such as cancer, psoriasis, arteriosclerosis, certain endocrine and metabolic disorders etc.

In the case of cancer, the IGF-1R is crucial for the transformation and proliferation of malignant cells. The IGF-1R is also important for preventing apoptosis and maintaining the malignant phenotype of tumour cells, and is involved in tumour cells developing resistance to the action of anti-cancer drugs. In contrast, the IGF-1R seems not to be an absolute requirement for normal cell growth.

The IGF-1R consists of two identical extracellular alpha-subunits that are responsible for ligand binding, and two identical beta-subunits with a transmembrane domain and an intracellular tyrosine kinase domain. The ligand-receptor interaction results in phosphorylation of tyrosine residues in the tyrosine kinase domain, which spans from amino acid 973 to 1229 of the beta-subunit. The major sites for phosphorylation are the clustered tyrosines at position 1131, 1135 and 1136 (LeRoith, D., et al., Endocr Rev 1995 April; 16(2), 143-63). After autophosphorylation, the receptor kinase phosphorylates intracellular proteins, like insulin receptor substrate-1 and Shc, which activate the phosphatidyl inositol-3 kinase and the mitogen-activated protein kinase signalling pathways, respectively.

Based on the pivotal role of IGF-1R in malignant cells, it becomes more and more evident that IGF-1R is a target for cancer therapy (Baserga, A, et al., Endocrine vol. 7, no. 1, 99-102, August 1997). A direct strategy to block IGF-1R activity is to induce selective inhibition of the IGF-1R tyrosine kinase. However, with the exception of our own recent discovery that certain cyclolignans (e.g. podophyllotoxin) and congeners can have this property (see below), no selective inhibitors of IGF-1R have been found.

Drugs containing the notoriously cytotoxic cyclolignan podophyllotoxin have been used for centuries, and its anti-cancer properties have attracted particular interest. However, undesired and severe side effects of podophyllotoxin have prevented its use as an anti-cancer drug. The mechanism for the cytotoxicity of podophyllotoxin has been attributed to its binding to beta-tubulin, leading to inhibition of microtubule assembly and mitotic arrest.

During the last decades the major interest in podophyllotoxin derivatives has concerned etoposide, which is an ethylidene glucoside derivative of 4′-demethyl-epipodophyllotoxin. Etoposide, which has no effect on microtubules, is a DNA topoisomerase II inhibitor, and is currently being used as such in cancer therapy.

### PRIOR ART

The IGF-1R is a member of the tyrosine kinase receptor family, which also includes the receptors of insulin, epidermal growth factor (EGF), nerve growth factor (NGF), and platelet-derived growth factor (PDGF). A number of synthetic tyrosine kinase inhibitors, called tyrphostins, have been studied by Párrizas, M., et al., Endocrinology 1997, Vol. 138, No. 4, 1427-1433. The major disadvantage with all of the tyrphostins active on IGF-1R is that they cross-react with the insulin receptor, since these receptors are highly homologous. However, some of the tyrphostins showed a moderate preference for IGF-1R, suggesting that it could be possible to design and synthesize small molecules capable of discriminating between these two receptors.

Substrate competitive inhibitors of the IGF-1 receptor kinase are discussed by Blum, G., et al. in Biochemistry 2000, 39, 15705-15712. A number of lead compounds for inhibitors of the isolated IGF-1R kinase are reported. The search for these compounds was aided by the knowledge of the three-dimensional structure of the insulin receptor kinase domain, which is 84% homologous to the IGF-1R kinase domain. One of the most potent inhibitors found was tyrphostin AG 538, with an IC₅₀ value of 400 nM. However, said inhibitor also blocked the insulin receptor kinase.

In WO 02/102804 A1and WO 02/102805 A1new compounds are disclosed, i.e. substituted 6-benzyl-1,3-benzodioxoles and substituted 1-phenyl-tetrahydro-naphtalenes, and the use thereof, as well as the use of certain cyclolignans as specific inhibitors of the insulin-like growth factor-1 receptor. Said compounds can be used for treatment of IGF-1R dependent diseases, especially cancer. The three-dimensional structures (folding) of short peptides having the amino acid sequence of the IGF-1R tyrosine domain, including the tyrosine residues at position 1131, 1135 and 1136, constructed by the computer, were studied in order to find compounds having the ability to mimick the tyrosine residues and thereby interfere with their phosphorylation. It was then discovered, when using a 12-amino acid peptide, that the hydroxy groups of two of the three key tyrosines, that is 1135 and 1136, which have to be autophosphorylated in IGF-1R for activation, could be situated as close as about 0.95 nm (9.5 Å) from each other, and that the apparent angle between these tyrosines was about 60°. Such a short distance for the corresponding tyrosines in the almost identical tyrosine domain of the insulin receptor had not previously been observed.

Molecular modelling showed that a molecule consisting of two benzene rings separated by only one carbon atom could mimick the suggested 3-dimensional structure of the two IGF-1R tyrosines. When a two-carbon bridge was tried, the distance between the substituents of the benzene rings seemed to be too long, about 1.3 nm (13 Å).

It was also presumed that the substituents of potential inhibitors' benzene rings, corresponding to the hydroxy groups in the IGF-1R tyrosines, should preferably be chemically relatively stable, e.g. methoxy or methylenedioxy groups, since these would not readily react and be transformed. The distance between such substituents also seemed to be roughly about 0.95 nm (9.5 Å).

This hypothesis led to the surprising discovery that podophyllotoxin and some other cyclolignans are potent and selective inhibitors of the IGF-1R by blocking tyrosine phosphorylation. In agreement with the hypothesis, these compounds have two angled benzene rings, which may, at least in theory, be able to mimick the two tyrosines 1135 and 1136 and/or fit into the tyrosine kinase pocket and thereby interfere with autophosphorylation of the tyrosines.

Before this discovery of ours, a connection between the IGF-1R and these compounds, including podophyllotoxin derivatives (cyclolignans), had not been made.

The Chemistry of Podophyllum by J. L. Hartwell et al., Fortschritte der Chemie organischer Naturstoffe 15, 1958, 83-166, gives an overview of podophyllotoxin and different derivatives thereof, which are commercially derived from two species of plants, Podophyllurn peltatum and Podophyllum emodi. As said, the cytotoxic effect of podophyllotoxin has been ascribed to its binding to microtubuli resulting in a mitotic block. The same effect on cells has been described for several of its derivatives.

The binding of certain 3-amino-substituted 1-phenyl-1,2,3,4-tetrahydronaphtalenes to a receptor with σ-like neuromodulatory activity in the mammalian central nervous system has been studied by Wyrick, S. D., et al., Journal of Medical Chemistry 36 (1993), 2542-2551.

Syntheses and structure-activity evaluation of a number of substituted benzyl-benzenes, also including 6-benzyl-1,3-benzodioxoles, have been carried out by L. Jurd (e.g S. C. Rawlins et al., J. Econ. Entomol. 72, 674-677, 1979; L. Jurd et al., S. Agric. Food Chem., 27, 1007-1016, 1979; L. Jurd, U.S. Pat. No. 4,342,777; L. Jurd, J. Heterocyclic Chem., 22, 993-995, 1984; J. K. Batra et al., Mol. Pharmacol., 27, 94-102, 1985; L. Jurd et al., J. Med.Chem., 30, 1752-1756, 1987; J. K. Batra et al., Biochem. Pharmac.35, 4013-4018, 1986) and more recently of benzophenones by G. R. Pettit (G. R. Pettit et al., J. Med. Chem., 41, 1688-1695, 1998). In the former studies the compounds were found to be active as insect chemosterilants and in the latter, the cytotoxic activity of derivatives of benzyl-benzenes having a structural similarity to podophyllotoxin was tested. The ability of the compounds to inhibit tubulin polymerisation was studied, but often compounds most similar to podophyllotoxin seemed to be the least active. The cytotoxicity of some benzopyrans and 4-aza-2,3-didehydro-podophyllotoxin has also been studied (J. K. Batra et al., Biochem.Pharmac., 37, 2595-2602, 1988; L. Jurd, J. Heterocyclic Chem., 33, 1227-1232, 1996; the patent application WO 00/04901, PCT/US99/12384) (C. Tratrat et al., Organic Letters, 4, 3187-3189, 2002; the European patent application EP 1 103 554 A1).

Although some of the mentioned compounds have been noted to possess some cytotoxic activity, the activity has never been associated to an inhibition of IGF-1R. In fact, their mechanism of action has not been characterized or has just been believed to be caused by a binding to microtubuli in analogy with that of podophyllotoxin, and therefore they are expected to be of limited usefulness. In one case, binding of substituted benzopyrans to the Bcl-2 protein was theoretically suspected but not tested (the patent application WO 00/04901, PCT/US99/12384).

### OBJECTS OF THE INVENTION

The object of the invention is to find new compounds and new methods for treatment of IGF-1/IGF-1R dependent diseases, such as cancer, psoriasis, arteriosclerosis, certain endocrine and metabolic disorders etc., by means of a specific inhibition of the insulin-like growth factor-1receptor.

### BRIEF DESCRIPTION OF THE DRAWINGS

FIG. 1 shows the 3-dimensional structure of the compound 4,5-demethylene-deoxypodophyllotoxin. For comparison, the stuctures of podophyllotoxin and the 12 amino acid peptide comprising the tyrosines 1131, 1135 an 1136 of the IGF-1 receptor, constructed by the computer, are also shown.

FIG. 2 shows the structural formulas of podophyllotoxin and 4,5-demethylene-deoxypodophyllotoxin.

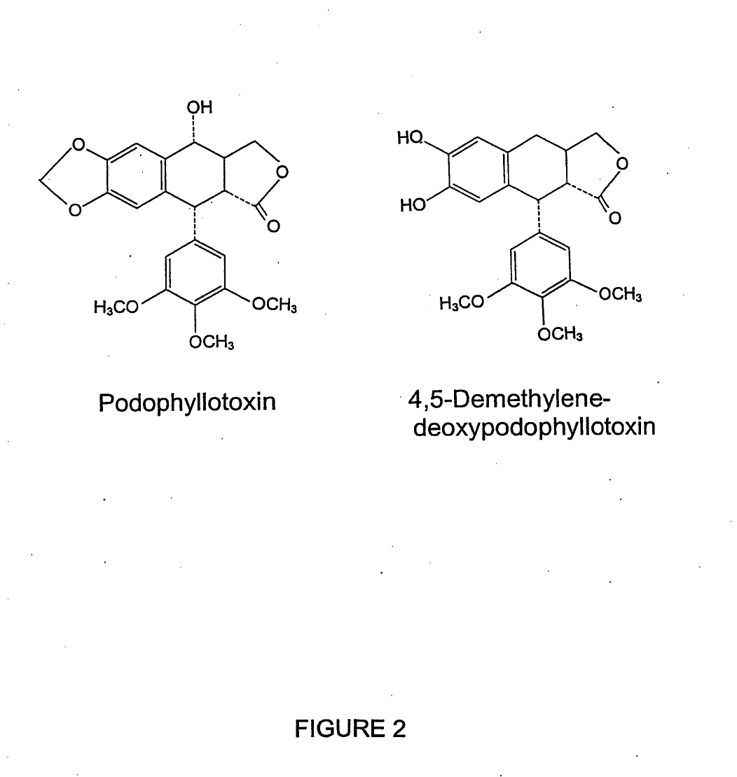

FIG. 3 shows the structural formulas of some substituted benzyl-benzenes representing compounds from group Ib.

FIG. 4 shows the structural formulas of some substituted 4-phenyl-chromans/chromens (benzopyranes) and 4-phenyl-tetrahydro/dihydro-quinolines (4-aza-2,3-didehydropodophyllotoxin) representing compounds from group II.

FIG. 5 shows the structural formulas of some substituted 4-phenyl-tetrahydronaphtalenes representing compounds from group III.

FIG. 6 shows the structural formulas of some cyclolignans representing compounds from group IV.

FIG. 7 shows the structural formulas of the cyclolignans picropodophyllin and beta-picropeltatin as phosphate and valerate esters representing compounds from group IV.

### DESCRIPTION OF THE INVENTION

Common to podophyllotoxin and all other previously found inhibitory cyclolignans was that one of the benzene rings was substituted with one methylenedioxy group (only) and the second benzene ring was substituted with either three methoxy groups or one hydroxy and two methoxy groups.

We have now surprisingly found that the substituents of the benzene rings of specific IGF-1R inhibitory molecules can also be different from those described in WO 02/102804 A1 and WO 02/102805 A1. For example, the methylenedioxy group can be replaced by two hydroxy groups. The substituents may also consist of methyl groups, halogens etc. and these new inhibitors of the IGF-1R are described here. This discovery opens the possibility to find additional specific IGF-1R inhibitors with advantageous properties. Thus, in addition to cyclolignans, other groups of compounds, which also may fit into the IGF-1R kinase pocket and mimick the tyrosines 1135 and 1136, have been found to be substituted benzyl-benzenes, benzophenones, 4-phenyl-chromans/chromens (benzopyran compounds), 4-phenyl-tetrahydro/dihydroquinolines (4-aza-2,3-didehydropodophyllotoxins) and substituted 4-phenyl-tetrahydronaphtalenes. Advantages with these compounds may be that they can be less toxic to normal cells than for example podophyllotoxin and derivatives. Furthermore, they may be pharmacologically more acceptable and/or easier to synthesize.

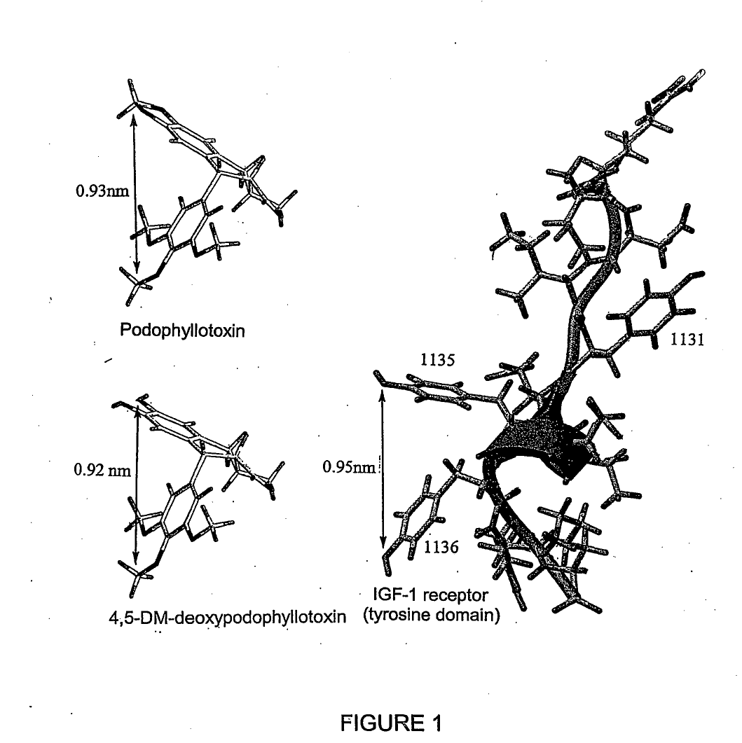

FIG. 1 shows the space structures of podophyllotoxin and the compound 4,5-demethylene-deoxypodophyllotoxin.

In order to penetrate the receptor and fit into the tyrosine kinase pocket, one can expect that an inhibitory molecule has to be small. When for instance podophyllotoxin was conjugated with a glucoside derivative, podophyllotoxin-4,6-O-benzylidene-β-D-glucopyranoside, the effect on IGF-1R completely disappeared. Furthermore, following reduction of the lactone ring to a diol structure, the size of the molecule increased due to the reduced substituents sticking out from the molecule, resulting in a dramatically reduced activity of the compound.

The inhibitory molecule also has to be relatively nonpolar, so that it can freely penetrate cell membranes and the IGF-1 receptor, but sufficiently polar to be reasonably soluble in water. The polarity of the molecule is determined by the number and nature of oxygen, nitrogen and some other functions. The polarity may be optimal when the water solubility is between 0.01 mM and 0.5 mM. Therefore charged or highly polar groups may decrease the potency of the molecule. On the other hand, a charged (polar) or a nonpolar (in some cases bulky) group may be coupled to the active inhibitor in order to improve its water solubility or prolong its time of release/action. In this case, the compound is a prodrug, which will be activated in the body by removal (e.g. by enzymes) of the mentioned group. Examples of such groups are phosphoric acid and fatty acids forming esters with a hydroxy group of the active molecule.

The invention refers to the uses and compounds as disclosed in the appended claims.

The invention refers to the use of a compound comprising the formula 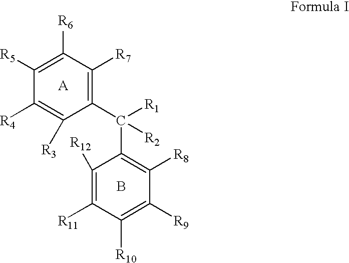
wherein the two benzene rings A and B are linked together by a carbon atom; the benzene rings A and B optionally may be replaced by the heterocyclic rings pyridine or pyrimidine; the carbon atom (bridge) between the two benzene rings may optionally be exchanged for a nitrogen, an oxygen or a sulphur atom; R₁ and R₂ are H, OH, C₁₋₆ linear or branched alkoxy chain or a C₁₋₂₀ linear or branched hydrocarbon (alkyl) chain, the mentioned alkoxy or alkyl chain optionally possessing 0-2 double bonds, 0-1 triple bond, 0-4 oxygen functions, 0-3 nitrogen-, 0-3 halogen- and 0-2 sulphur-containing substituents, 0-2 phosphate groups (OPO₃), 0-2 nonsubstituted or substituted phenyl or cyclohexyl groups, 0-2 five- or six-member heterocyclic rings; one of the above mentioned alkyl or alkoxy chains may optionally form a bond with a carbon atom in the benzene ring A (instead of the R₇ substituent) via a carbon-, oxygen-, nitrogen- or sulphur- atom; or R₁ and R₂ can together form a double bond to the above mentioned alkyl chain, to an oxo group, to a sulphur atom or to a nitrogen atom substituted with H, OH, an alkyl or alkoxy group. Oxygen functions in this context refer to for example hydroxy, oxo, aldehyde, carboxy, alkoxy, O(CH₂)₁₋₃O, OCHCH₃O, dimethylmethylenedioxy (acetonide), carbonyldioxy (carbonate), lactone, ether and/or ester (OCOH, OCO(CH₂)₀₋₁₈CH₃) groups. Nitrogen containing substituents in this context refer to for example NH₂, NH(C₁₋₃ alkyl), N(C₁₋₃ alkyl)₂, NO₂, NHCOCH₃, NHNHCOCH₃, NHNHCONH₂, NHCH₂CH₂, NHCOCH₂, CN, CH₂CN, CH₂NH₂, CH₂NO₂, CONH₂, CONHCH₃, CONHNHCH₂CH₃, NH, NCH₃, NOH, NOCH₃ and NOC₂H₅. Halogen containing substituents in this context refer to for example F, Cl, Br, I and CF₃. Sulphur containing substituents in this context refer to for example SCH₃, S (sulphide) and SO₂ (sulphone). Five- and six-member heterocyclic rings are preferably pyrrolyl, pyrrolidino, imidazyl, furyl, tetrahydrofuryl, piperidinyl, pyridinyl, pyrimidyl, pyryl, tetrahydropyryl, morpholino and piperazinyl.

The benzene ring substituents R₃, R₄, R₅, R₆, R₇, R₈, R₉, R₁₀, R₁₁ and R₁₂ and the substituents of the phenyl group mentioned above, which can be the same or different, are H, OH, OCH₃, OCH₂CH₃, OCH₂CH₂CH₃, OCH(CH₃)₂, OC(CH₃)₃, OCHCH₂, OCHCHCH₃, OCH₂CHCH₂,OCCH, OCOH, OCO(CH₂)₀₋₁₈CH₃, OCH₂OH, OCHO, OCOOH, OCOCH₃, OCOC₂H₅, OCOC₃H₇, OCOOCH₃, OCOOC₂H₅, OCOOC₃H₇, OCH₂OOCH, OCH₂OOCCH₃, OCH₂OOCC₂H₅, OCH₂CH₂OH, OCH₂CHO, OCH₂COOH, OC₂H₄CH₂OH, OC₂H₄CHO, OC₂H₄COOH, CH₃, CH₂CH₃, CH₂CH₂CH₃, CH(CH₃)₂, C(CH₃)₃, CHCH₂, CHCHCH₃, CH₂CHCH₂, CCH, CH₂OH, CHO, COOH, COCH₃, COC₂H₅, COC₃H₇, COOCH₃, COOC₂H₅, COOC₃H₇, CH₂OOCH, CH₂OOCCH₃, CH₂OOCC₂H₅, CH₂CH₂OH, CH₂CHO, CH₂COOH, C₂H₄CH₂OH, C₂H₄CHO, C₂H₄COOH, F, Cl, Br, I, CF₃, CN, NH₂, NO₂, CH₂CN, CH₂NH₂, CH₂NO₂, CONH₂, CONHCH₃, NH(C₁₋₃ alkyl), N(C₁₋₃ alkyl)₂, NHCOCH₃, NHNHCOCH₃, NHNHCONH₂, SCH₃, OPO₃ and/or OSi(CH₃)₂C(CH₃)₃.

Two substituents on adjacent carbons in the benzene rings may together form the group CH₂CH₂CH₂, or CH₂CH₂CH₂CH₂ or O(CH₂)₁₋₃O, OCHCH₃O, OC(CH₃)₂O,OOCO (carbonyldioxy=carbonate), OCOCH₂, NHCH₂CH₂, NHCOCH₂.

When the compound includes at least one charged group, e.g. NH₂, COOH or OPO₃, a pharmaceutically acceptable salt thereof will be formed.

The above description of structures is valid with the proviso that when R₄ and R₅ together form a methylenedioxy group in the A-ring, then I: not all of the other substituents in this ring are hydrogens or when R₇ forms a bond with the alkyl chain described below, both R₃ and R₆ are not hydrogens; or when R₄ and R₅ together form a methylenedioxy group in the A-ring and when I is not valid, then II: the substituents of the B-ring (excluding hydrogens) are not 1-3 methoxy groups, 1 methoxy group and 1-2 hydroxy groups or 2 methoxy groups and 1 hydroxy group; or when R₄ and R₅ together form a methylenedioxy group in the A-ring and I and II are not valid, and R₂ is a hydrogen, then III: R₁ is not H, OH, OCH₃, OC₂H₅, or a C₁₋₅ linear or branched hydrocarbon chain with 0-1 double bond and forming or not forming a bond with a carbon atom in the A-ring (corresponding to the substituent R₇) and with 0-3 oxygen functions; or when R₄ and R₅ together form a methylenedioxy group in the A-ring and I, II and III are not valid, and R₂ is a hydrogen, then IV: the distance between the carbon atom of the methylenedioxy group and the carbon atom of a methoxy group in the B-ring is not 0.85-1.05 nm.

Oxygen functions in this context refer to hydroxy, oxo, carboxy, methoxy, methylenedioxy, lactone, ether acetonide, carbonate and/or ester groups.

The invention refers to the use of such compounds as specific inhibitors of tyrosine phosphorylation of the insulin-like growth factor-1 receptor.

One group of compounds which can be used in accordance with the invention has the formula Ib 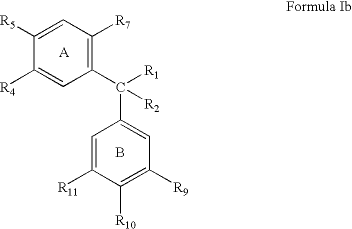
wherein R₁ and R₂, which can be the same or different, are preferably H, OH, CH₃, CH₂CH₃, CH₂CH₂CH₃, CH₂CH₂CH₂CH₃,CH₂CHCH₂, CH₂CH(CH₃)₂, OCH₃, OCH₂CH₃, OCH₂CH₂CH₃, OCH₂CH₂CH₂CH₃, OCH₂CHCH₂, OCH₂CH(CH₃)₂, CH₂OH, CH₂CH₂OH, CH₂CH₂CH₂OH, OCH₂OH, OCH₂CH₂OH, OCH₂CH₂CH₂OH, a phenyl or piperidinyl or morpholino group; R₁ and R₂, when together, are preferably O, CH₂, CHCH₃, CHCH₂CH₃, C(CH₃)₂, CHCH(CH₃)₂, C(CH₂CH₃)phenyl, NOH, NOCH₃, NOCH₂CH₃, NOCH₂CH₂CH₃.

The phenyl substituents R₄, R₅, R₇, R₉, R₁₀ and R ₁₁ which may be the same or different, are preferably H, OH, CH₃, CH₂CH₃, CH₂CH₂CH₃, CH₂CHCH₂, OCH₃, OCH₂CH₃, OCH₂CH₂CH₃, OCH₂CHCH₂, CH₂OH, CH₂CH₂OH, OCH₂OH, OCH₂CH₂OH, COOCH₃, F, Cl, CF₃, NH₂ and NHCH₃, OCO(CH₂)₀₋₁₈CH₃ and OPO₃; the adjacent substituents R₄ and R₅ and/or R₉ and R₁₀ may together form preferably a methylenedioxy group.

The above description of structures is valid with the proviso that when R₄ and R₅ together form a methylenedioxy group in the A-ring, then I: R₇ is not a hydrogen; or when R₄ and R₅ together form a methylenedioxy group in the A-ring and when I is not valid, then I: the substituents of the B-ring (excluding hydrogens) are not 1-3 methoxy groups, 1 methoxy group and 1-2 hydroxy groups or 2 methoxy groups and 1 hydroxy group; or when R₄ and R₅ together form a methylenedioxy group in the A-ring and I and II are not valid, and R₂ is a hydrogen, then III: R₁ is not H, OH, OCH₃, OC₂H₅, CH₃, C₂H₅ or C₂H₄OH; or when R₄ and R₅ together form a methylenedioxy group in the A-ring and I, II and III are not valid, and R₂ is a hydrogen, then IV: the distance between the carbon atom of the methylenedioxy group and the carbon atom of a methoxy group in the B-ring is not 0.85-1.05 nm.

The syntheses of representative compounds of the formula Ib have been described previously (L. Jurd et al., J. Agric. Food Chem., 27, 1007-1016, 1979; L. Jurd, U.S. Pat. No. 4,342,777; L. Jurd, J. Heterocyclic Chem., 22, 993-995, 1984; L. Jurd et al., J. Med.Chem., 30, 1752-1756, 1987; G. R. Pettit et al., J. Med. Chem., 41, 1688-1695, 1998; U.S. Pat. No. 2,825,730; the European patent application: EP 0 781 749 A2).

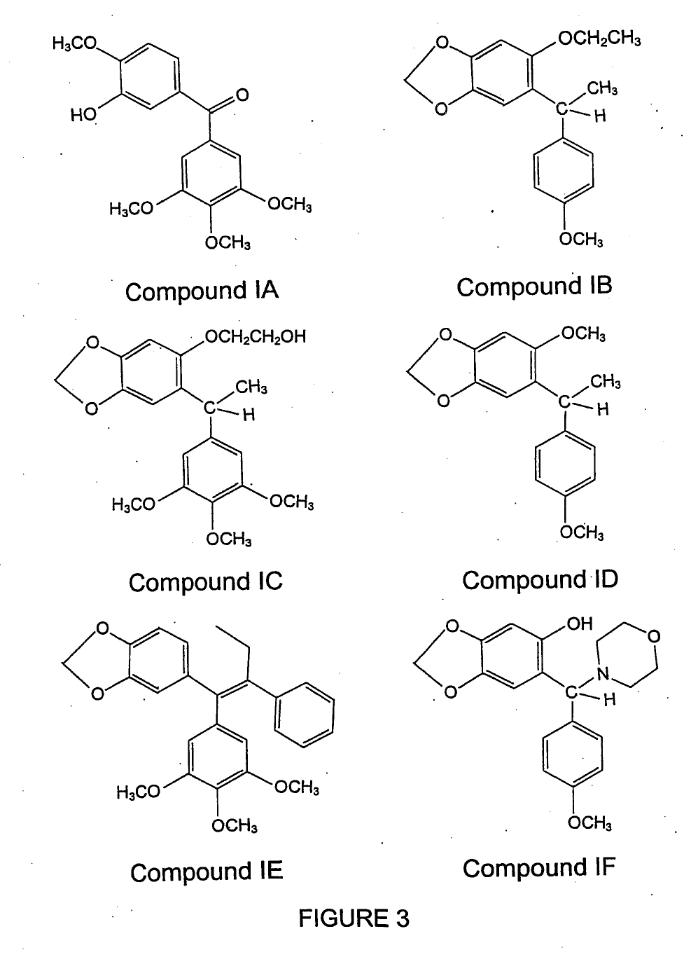

Examples of representative compounds of the formula Ib (substituted benzyl-benzenes), which can be prepared in this way are illustrated in FIG. 3.

Another group of compounds which can be used in accordance with the invention has the formula II 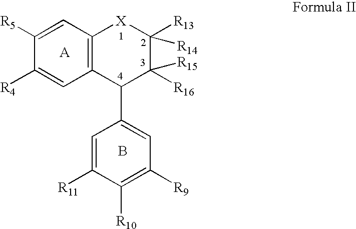
wherein X can be O, NH, NCH₃, NCH₂CH₃, NOH, NOCH₃, S or SO₂; optionally there is a double bond between carbon 2 and 3 and then the substituents R₁₄ and R₁₅ are absent; the substituents R₁₃, R₁₄, R₁₅ and R₁₆, which can be the same or different, are preferably H, OH, CH₃, CH₂CH₃, CH₂CH₂CH₃, CH₂CH₂CH₂CH₃, CH₂CHCH₂, CH₂CH(CH₃)₂, OCH₃, OCH₂CH₃, OCH₂CH₂CH₃, OCH₂CH₂CH₂CH₃, OCH₂CHCH₂, OCH₂CH(CH₃)₂, CH₂OH, CH₂CH₂OH, CH₂CH₂CH₂OH, OCH₂OH, OCH₂CH₂OH, OCH₂CH₂CH₂OH, a phenyl or piperidinyl or morpholino group; R₁₃ and R₁₄ together or R₁₅ and R₁₆ together are preferably O or R₁₄ and R₁₅ together is preferably CH₂OCO or COOCH₂(lactone rings), CH₂OCH₂(ether), CH₂CH₂CO, CH₂OC(CH₃)₂OCH₂, OC(CH₃)₂O (acetonide), CH₂OCOOCH₂, OCOO (carbonate), CH₂OCH₂OCH₂ or OCH₂O (methylendioxy) group.

The phenyl substituents R₄, R₅, R₉, R₁₀ and R₁₁, which may be the same or different, are preferably H, OH, CH₃, CH₂CH₃, CH₂CH₂CH₃, CH₂CHCH₂, OCH₃, OCH₂CH₃, OCH₂CH₂CH₃, OCH₂CHCH₂, CH₂OH, CH₂CH₂OH, OCH₂OH, OCH₂CH₂OH, COOCH₃, F, Cl, CF₃, NH₂ and NHCH₃, OCO(CH₂)₀₋₁₈CH₃ and OPO₃; the adjacent substituents R₄ and R₅ and/or R₉ and R₁₀ may together form preferably a methylenedioxy group.

Compounds of the formula II can be prepared by the representative syntheses reported previously (L. Jurd, J. Heterocyclic Chem., 33, 1227-1232, 1996; C. Tratrat et al., Organic Letters, 4,3187-3189,2002; the European patent application EP 1 103 554 A1) and a large number, which are contained in the Available Chemicals Dictionary (Molecular Design Limited, San Leonardo, Calif.), are also commercially available.

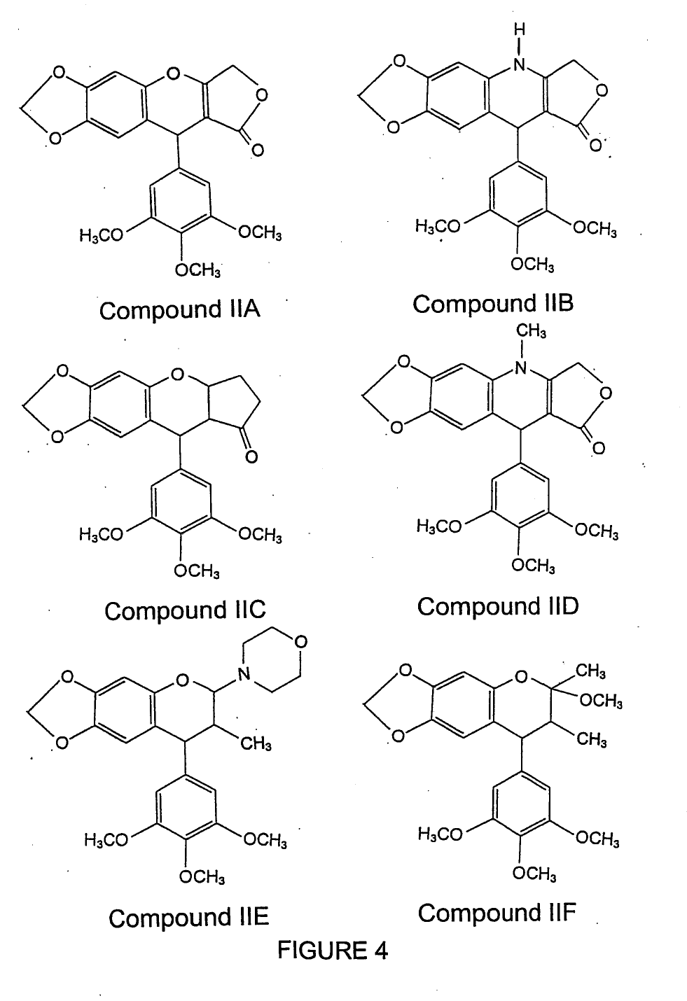

Examples of representative compounds of the formula II (substituted 4-phenyl-chromans/chromens and 4-phenyl-1,2,3,4-tetrahydro-quinolines/1,4-dihydroquinolines), which can be prepared in this way are illustrated in FIG. 4.

Still another group of compounds which can be used in accordance with the invention has the formula III 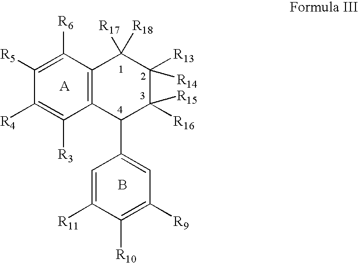
wherein optionally there is a double bond between carbon 2 and 3 and then the substituents R₁₄ and R₁₅ are absent; the substituents R₁₃, R₁₄, R,₁₅,R₁₆, R₁₇ and R₁₈, which can be the same or different, are preferably H, OH, OCH₃, OCH₂CH₃, OCH₂CH₂CH₃, OCH₂CH₂CH₂CH₃, OCH₂CHCH₂, OCH₂CH(CH₃)₂, CH₂OH, CH₂CH₂OH, CH₂CH₂CH₂OH, OCH₂OH, OCH₂CH₂OH, OCH₂CH₂CH₂OH, a phenyl or piperidinyl or morpholino group; R₁₅ and/or R₁₆ and R₁₇ and R₁₈ can also preferably be CH₃, CH₂CH₃, CH₂CH₂CH₃, CH₂CH₂CH₂CH₃, CH₂CHCH₂, CH₂CH(CH₃)₂; R₁₃ and R₁₄ together or R₁₄ and R₁₅ together or R₁₇ and R₁₈ together are preferably 0; R₁₄ and R₁₅ together can be OC(CH₃)₂O (acetonide), OCOO (carbonate) or OCH₂O (methylendioxy) group. The phenyl substituents R₃, R₄, R₅, R₆, R₉, R₁₀ and R₁₁, which may be the same or different, are preferably H, OH, CH₃, CH₂CH₃, CH₂CH₂CH₃, CH₂CHCH₂, OCH₃, OCH₂CH₃, OCH₂CH₂CH₃, OCH₂CHCH₂, CH₂OH, CH₂CH₂OH, OCH₂OH, OCH₂CH₂OH, COOCH₃, F, Cl, CF₃, NH₂ and NHCH₃I OCO(CH₂)₀₋₁₈CH₃ and OPO₃; the adjacent substituents R₄ and R₅ and/or R₉ and R₁₀ may together form preferably a methylenedioxy group.

The above description of structures is valid with the proviso that when R₄ and R₅ together form a methylenedioxy group in the A-ring, then I: both R₃ and R₆ are not hydrogens; or when R₄ and R₅ together form a methylenedioxy group in the A-ring and when I is not valid, then II: the substituents of the B-ring (excluding hydrogens) are not 1-3-methoxy groups, 1 methoxy group and 1-2 hydroxy groups or 2 methoxy groups and 1 hydroxy group; or when R₄ and R₅ together form a methylenedioxy group in the A-ring and I and II are not valid, then III: together R₁₃ and R,₁₄, R₁₅ and R₁₆ or R₁₇ and R₁₈ is not an oxo group or when R₁₃, R₁₅ and R₁₇ are hydrogens, R₁₄, R₁₆ and R₁₈ is not only H, OH or OCH₃; or when R₄ and R₅ together form a methylenedioxy group in the A-ring and I, II and m are not valid, then IV: R₁₃ and R₁₇ or R₁₃ and R₁₅ do not together form a methylenedioxy group, acetonide (dimethyl-methylenedioxy) group or a carbonate (carbonyldioxy) group; or when R₄ and R₅ together form a methylendioxy group in the A-ring and I, II, III and IV are not valid, then V: the distance between the carbon atom of the methylenedioxy group and the carbon atom of a methoxy group in the B-ring is not 0.85-1.05 nm.

Compounds of the formula III may be prepared according to representative syntheses depicted in Schemes 1 and 2: 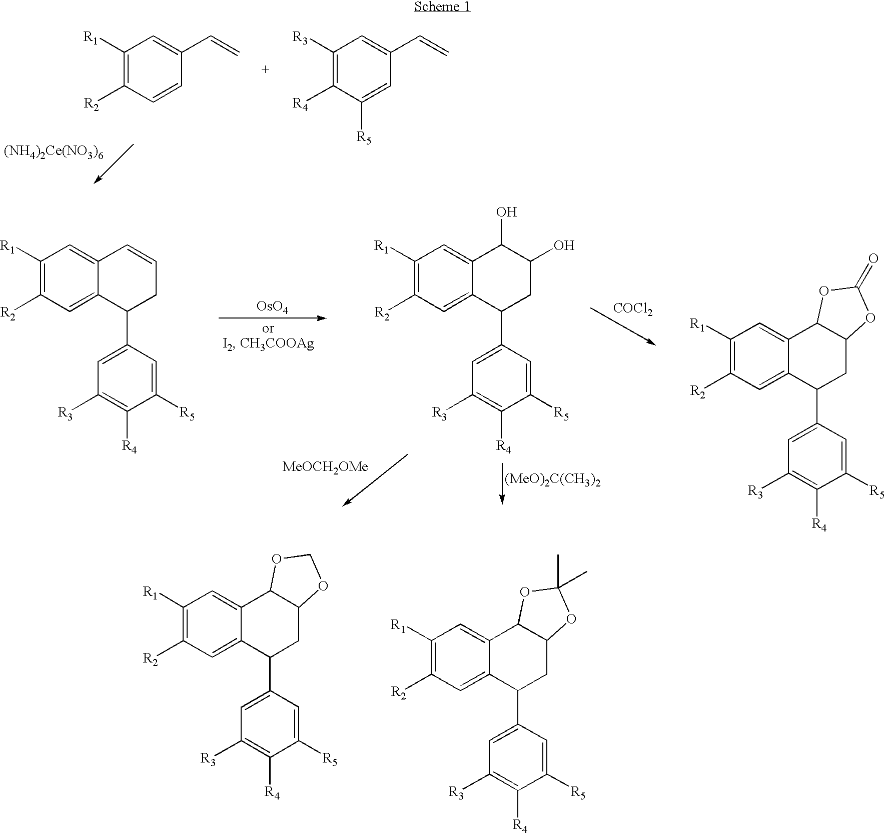
1-Phenyl-1,2-dihydronaphtalenes are known, see for example: Nair, V., et al.: Tetrahedron Letters (1977), 38(12), 2191-2194. Hydroxylation with OsO₄ gives the cis-hydroxyl product, whereas I₂/CH₃COOAg give trans. Stereoisomers will also be obtained relative to the phenyl group, depending on from which side the hydroxylation reagent attacks. This is also true for scheme 2 below. All such isomers are meant to be included in the reaction schemes. 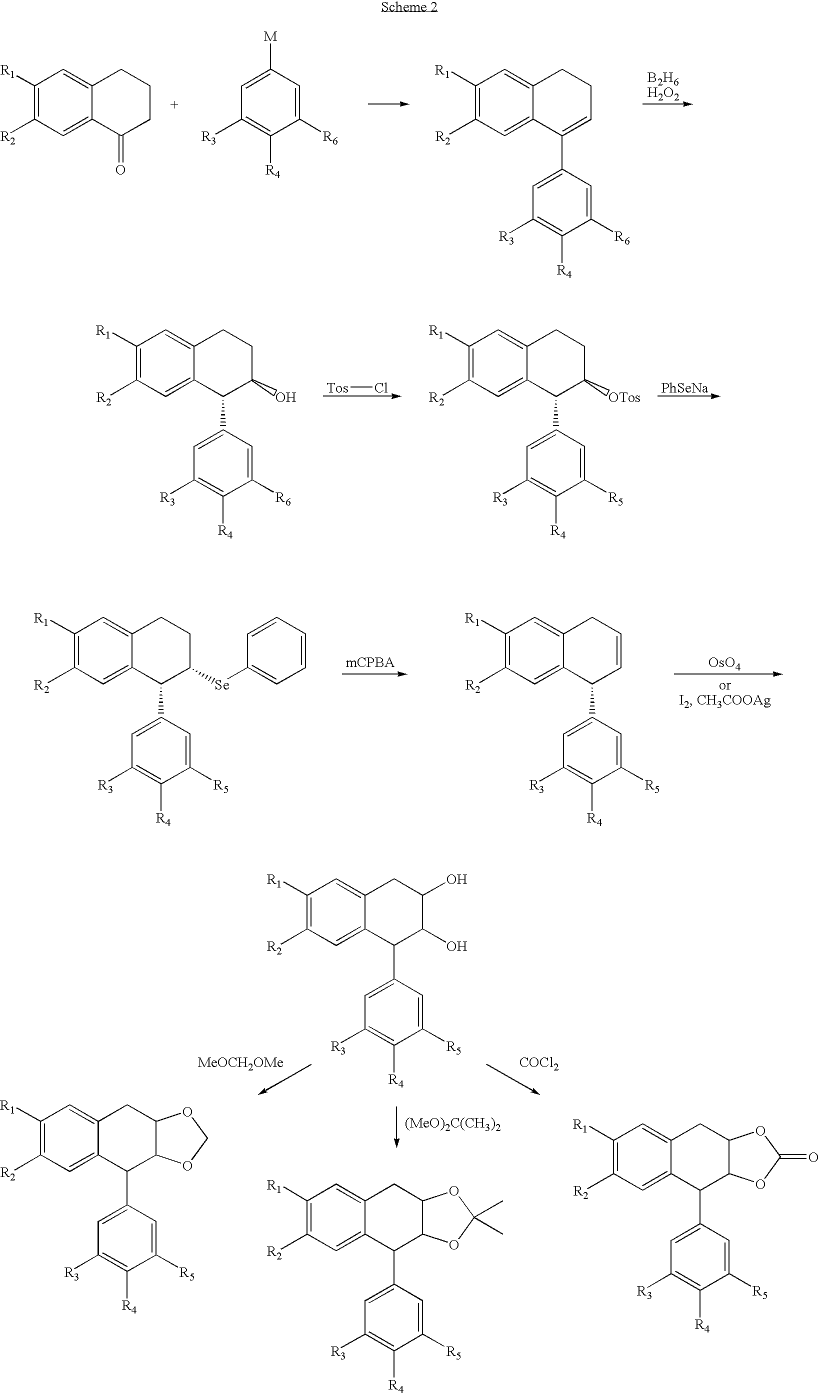
The first steps in this synthesis have been described in Heterocycles (1984), 22(2), 311-31 by G. Laus et al. The reactions outlined above are well known in the art, see e.g. Advanced Organic Chemistry, Jerry March (ed.) 4^{th} edition, Wiley-Interscience Publication, New York, 1992.

Compounds of the formula III may also be prepared according to representative syntheses depicted in Schemes 3 and 4: 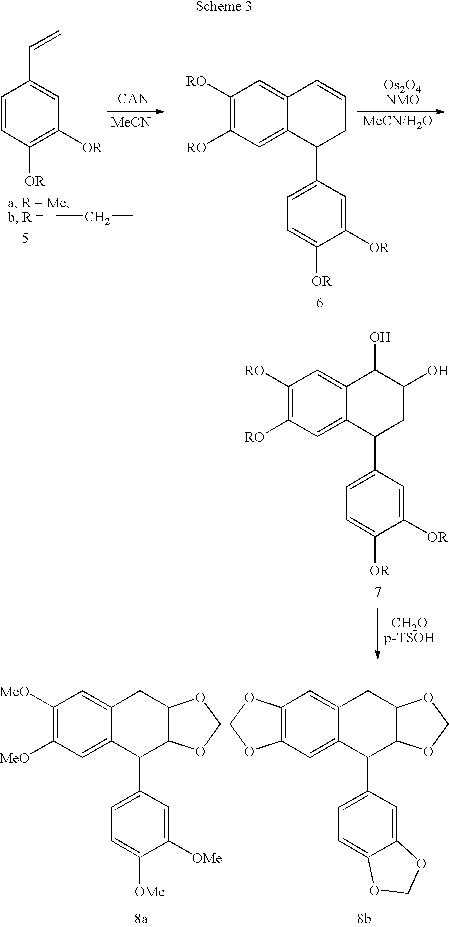
Compound 8a and 8b is formed in a three-step reaction starting from different substituted styrene derivatives (compound 5a and 5b).
D; By treating the styrene derivatives with cerium ammonium nitrate (CAN) in acetonitrile (MeCN) at low temperature in accordance with the literature (V. Nair, et al. Tetrahedron Lett. 1997, 38(12), 2191-2194) compounds 6a and 6b are formed.
E; Compounds 7a and 7b could then be formed from compounds 6a and 6b by oxidation of the double bond with OsO₄ and N-methyl morpholine oxide and MeCN/H₂O (P. Zubaidha et al. Tetrahedron, 1991, 47(30), 5759-5768.)
F; Finally, compounds 8a and 8b are formed by treatment of compounds 7a and 7b with formaldehyde under acidic conditions (p-toluene sulphonic acid), to form the methylene acetal (M. Anteunis et al. Synthesis, 1974, 23-26). 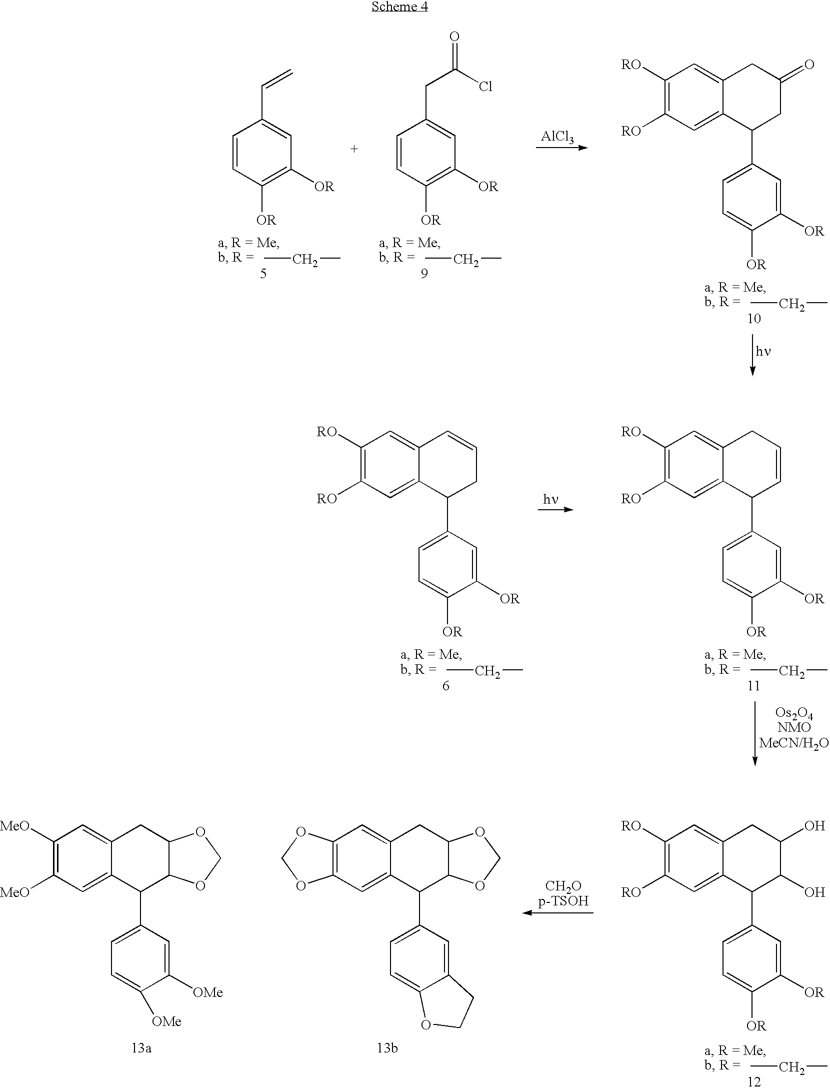
   Compounds 13a and 13b could be synthesized from compounds 11 in the same way as described above for the transformation from compound 6 to 8.
   Compound 11 could be formed in two ways:
a, The double bond in compound 6 could be photochemical rearranged according to xxx Rec. Trav. Chim. Pays-Bas, 1990, 109(3), 168-171.
b, Compound 11 could also be formed in a two-step reaction starting from styrene derivatives (5) and benzylic acid chorides (9) using the Lewis acid, AlCl₃ as reagent to form compound 10 in accordance with I. Fleming et al, J. Chem. Soc. Perkin Trans. 1, 1980, 11, 2485-2489. Compound 10 then form compound 11 under photochemical conditions. (J. J. Lamberts et al, J. Org. Chem. 1983, 48(13) 2202-2206).

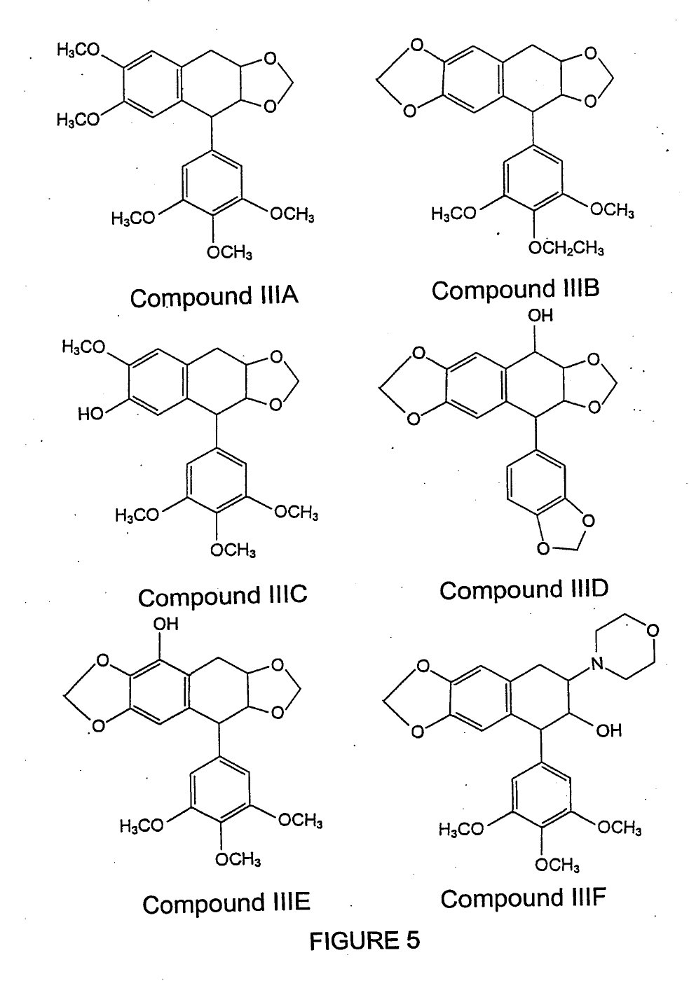

Examples of compounds of the formula III (4-phenyl-1,2,3,4-tetrahydro-naphtalenes/1,4-dihydronaphtalenes), which can be prepared in these ways are illustrated in FIG. 5.

Still another group of compounds which can be used in accordance with the invention are compounds of the formula IV 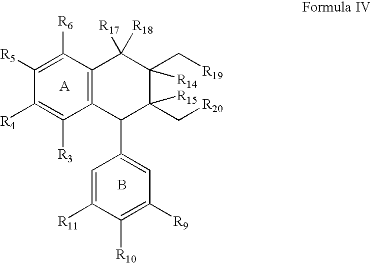
wherein optionally there is a double bond present so that either the substituents R₁₄ and R,₁₅ or R₁₄ and R₁₇ are absent; R₁₄ and R₁₅, which can be the same or different, are H, OH, CH₃ or OCH₃; R₁₇ and R₁₈, which can be the same or different, are H, OH, CH₃, CH₂CH₃, OCOH, OCO(CH₂)₀₋₁₈CH₃, OCH₃, OC₂H₅ and OPO₃; R₁₇ and R₁₈ together or R₁₉ or R₂₀ are preferably O, CH₂, CHCH₃, NOH, NOCH₃, NOCH₂CH₃; R₁₉ and R₂₀, which can be the same or different, are H, OH, OCH₃, or OC₂H₅, OOCH₃, OOCH₂CH₃, OCOH, OCO(CH₂)₀₋₁₈CH₃,; or R₁₉ and R₂₀ together form preferably a methylene bridge, an ether or a lactone group; the phenyl substituents R₃, R₄, R₅, R₆, R₉, R₁₀ and R₁₁, which may be the same or different, are preferably H, OH, CH₃, CH₂CH₃, CH₂CH₂CH₃, CH₂CHCH₂, OCH₃, OCH₂CH₃, OCH₂CH₂CH₃, OCH₂CHCH₂, CH₂OH, CH₂CH₂OH, OCH₂OH, OCH₂CH₂OH, COOCH₃, F, Cl, CF₃, NH₂ and NHCH₃, OCO(CH₂)₀₋₁₈CH₃ and OPO₀₃; the adjacent substituents R₄ and R₅ and/or R₉ and R₁₀ may together form preferably a methylenedioxy group.

The above description of structures is valid with the proviso that when R₄ and R₅ together form a methylenedioxy group in the A-ring, then I: both R₃ and R₆ are not hydrogens; or when R_{4 and R}₅ together form a methylenedioxy group in the A-ring and when I is not valid then II: the substituents of the B-ring (excluding hydrogens) are not 1-3 methoxy groups, 1 methoxy group and 1-2 hydroxy groups or 2 methoxy groups and 1 hydroxy group; or when R₄ and R₅ together form a methylenedioxy group in the A-ring and I and II are not valid, then III: together R₁₇ and R₁₈ are not an oxo group or when R₁₄, R₁₅ and R₁₇ are hydrogens, then R₁₈, R₁₉ and R₂₀ are not only H, OH, OCH₃ or OC₂H₅, or R₁₉ and R₂₀ are not OOCH₃ or OOCH₂CH₃ or together R₁₉ and R₂₀ do not form an ether or a lactone group; or when R₄ and R₅ together form a methylenedioxy group in the A-ring and I, II and III are not valid, then IV: the distance between the carbon atom of the methylenedioxy group and the carbon atom of a methoxy group in the B-ring is not 0.85-1.05 nm.

The invention especially refers to the use of relatively non-toxic cyclolignans as inhibitors of tyrosine autophosphorylation of the insulin growth factor-1 receptor, whereas the-use of the notoriously cytotoxic and tissue irritating compounds, such as podophyllotoxin and 4′-demethyl-podophyllotoxin should be avoided.

Some compounds of the formula IV are naturally occurring in plants, such as e.g. beta-peltatin (Podophyllum peltatum), austrobailignan 1 and 3 (Austrobaileya scandens) and polygamatin (Polygala polygama) as summarized by D. C. Ayres and J. D. Loike in Lignans. Chemical, biological and clinical properties (Cambridge University Press, Cambridge, pp. 12-84,1990).

For the preparation of said substances in pure form, dried and finely ground plant parts (e.g. rhizomes of Podophyllum peltatum) are extracted with organic solvents. The extract is then filtered and concentrated on silica gel. The fractions containing the substance(s) are collected and the latter is further purified by chromatography on acid alumina and silica gel etc., and finally recrystallized.

Naturally occurring but toxic cyclolignans, such as podophyllotoxin, beta-peltatin etc., having a lactone-ring with a trans-configuration, may be used as the starting material for the syntheses of their less toxic picro derivatives, i.e. those having a lactone-ring with a cis-configuration. Most cis picro) derivatives can be prepared from the trans compounds by this general synthesis: One mg of the trans compound is dissolved in 70% aqueous methanol. To the solution is added 20 mg of sodium acetate and the mixture is then incubated for 20 h at 55° C. After evaporation of the alcohol, the product is extracted with ethyl acetate, and then purified by chromatography on an open or HPLC column of silica gel using a mobile phase of hexane-ethyl acetate or hexane-isopropanol mixtures, and/or on a column of octadecylsilane-bonded silica using a mobile phase of aqueous methanol.

Compounds of the formula IV may also be prepared from 4′-demethyl-podophyllotoxin (1) or podophyllotoxin (2) according to the syntheses depicted in Scheme 5: 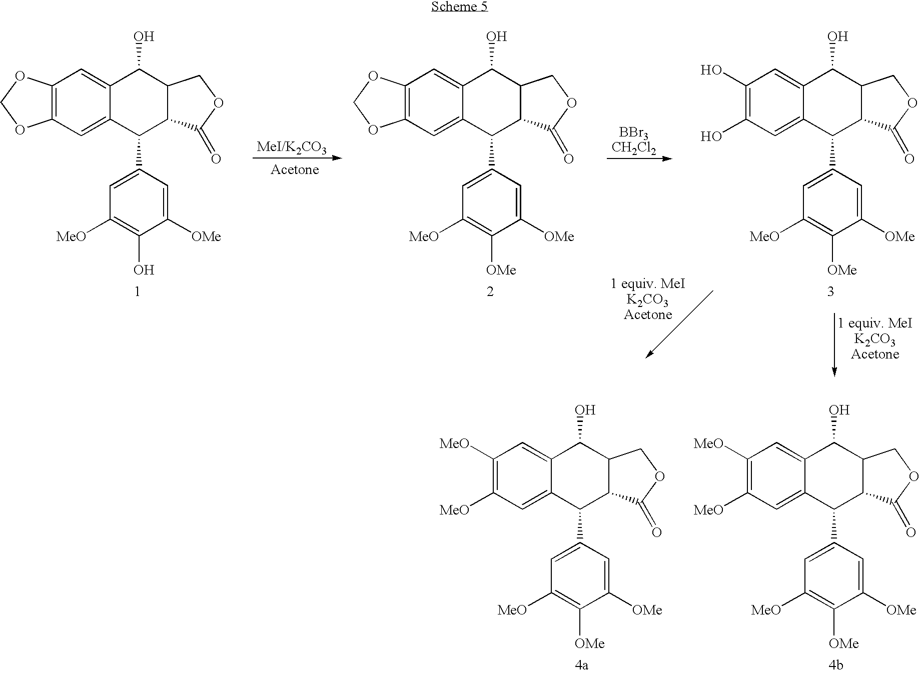
A; Selective O-methylation of phenolic hydroxyl in the presence of a secondary alcohols could be achieved by treatment of the starting material 1 with MeI and K₂CO₃ in acetone (D. Ma et al, Bioorg. Med. Chem. Lett. 2001, 11, 99-101). The lactone ring is under this mild basic condition stable.
B; Cleavage of the acetal ring, to form compounds 3 is achieved by treatment of compounds 2 with BBr₃ in methylene chloride in −78° C. to room temperature. The reaction is followed by TLC analysis and completion the temperature is lowered to −78° C. and the reaction is quenched by addition of methanol to the reaction mixture. This method is in accordance with S-Y. Sit et al. J. Med. Chem. 2002, 45, 3660-3668.
C; Compound 4a is formed from compound 3 in the same way ass described above for the formation of compound 2. Compound 4b could be formed by addition of only 1 equiv. MeI to compound 3,

The yield however, will be low since a mixture of different products will be formed.

The acetate (and other fatty acid esters) derivatives of cyclolignans can be prepared from the compound by incubating 0.1 mg of the latter with 1 mL of acetic anhydride (or corresponding fatty acid anhydride) and 1 mL of pyridine at 50° C. for 16 hours. The reagents are then partly evaporated, 10 mL of water and 10 mL of ethyl acetate are added and the product is then extracted from the aqueous phase.

Acetonides and methylenedioxy derivatives can be prepared starting from cyclolignans possessing two hydroxy groups (diols) obtained e.g. by reducing the lactone ring of natural lignans, e.g by LiAlH₄, according to standard procedures.

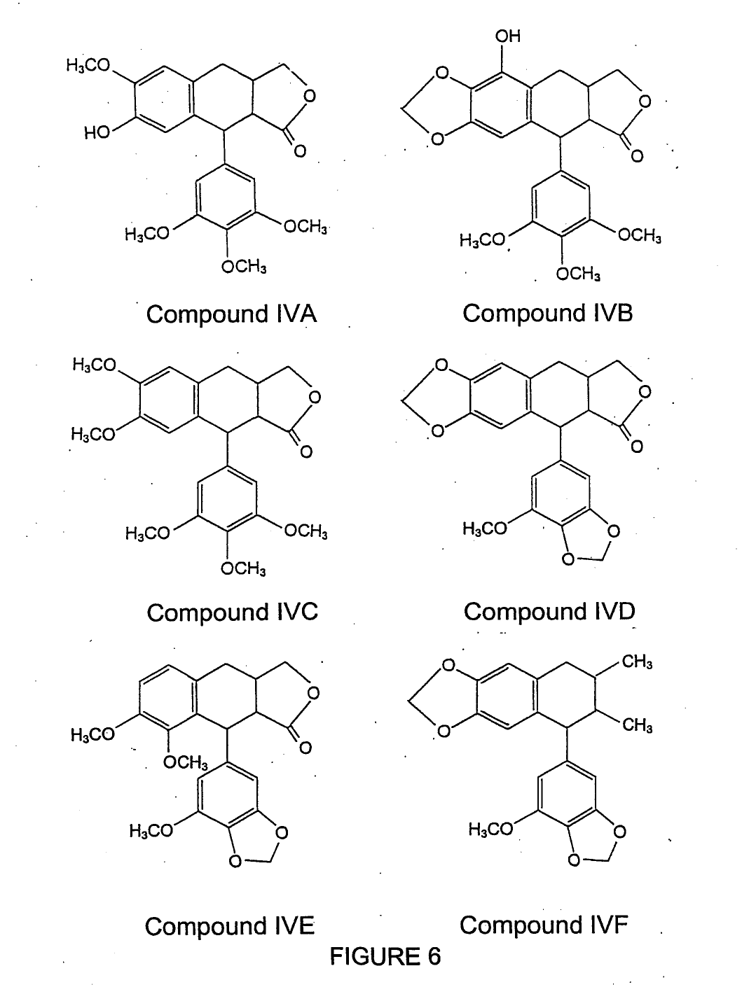

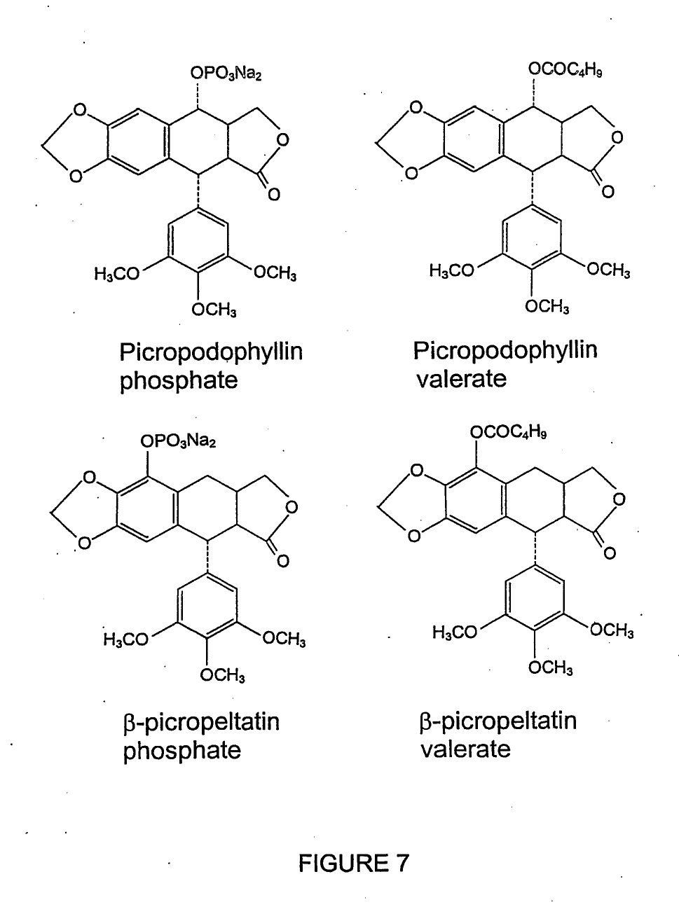

Examples of compounds of the formula IV (cyclolignans), which can be isolated/prepared in these ways are illustrated in FIG. 6 and FIG. 7.

The invention also refers to the new compounds of the formula III 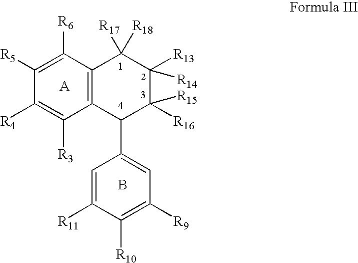
wherein optionally there is a double bond between carbon 2 and 3 and then the substituents R₁₄ and R₁₅ are-absent; the substituents R₁₃, R₁₄, R₁₅, R₁₆, R,₁₇ and R₁₈, which can be the same or different, are preferably H, OH, OCH₃, OCH₂CH₃, OCH₂CH₂CH₃, OCH₂CH₂CH₂CH₃, OCH₂CHCH₂, OCH₂CH(CH₃)₂, CH₂OH, CH₂CH₂OH, CH₂CH₂CH₂OH, OCH₂OH, OCH₂CH₂OH, OCH₂CH₂CH₂OH, a phenyl or piperidinyl or morpholino group; R₁₅ and/or R₁₆ and R₁₇ and R₁₈ can also preferably be CH₃, CH₂CH₃, CH₂CH₂CH₃, CH₂CH₂CH₂CH₃, CH₂CHCH₂, CH₂CH(CH₃)₂; R₁₃ and R₁₄ together or R₁₄ and R₁₅ together or R₁₇ and R₁₈ together are preferably O; R₁₄ and R₁₅ together can be OC(CH₃)₂O (acetonide), OCOO (carbonate) or OCH₂O (methylendioxy) group. The phenyl substituents R₃, R₄, R₅, R₆, R₉, R₁₀ and R ₁₁, which may be the same or different, are preferably H, OH, CH₃, CH₂CH₃, CH₂CH₂CH₃, CH₂CHCH₂, OCH₃, OCH₂CH₃, OCH₂CH₂CH₃, OCH₂CHCH₂, CH₂OH, CH₂CH₂OH, OCH₂OH, OCH₂CH₂OH, COOCH₃, F, Cl, CF₃, NH₂ and NHCH₃, OCO(CH₂)₀₋₁₈CH₃ and OPO₃; the adjacent substituents R₄ and R₅ and/or R₉ and R₁₀ may together form preferably a methylenedioxy group.

The above description of structures is valid with the proviso that when R₄ and R₅ together form a methylenedioxy group in the A-ring, then I: both R₃ and R₆ are not hydrogens; or when R₄ and R₅ together form a methylenedioxy group in the A-ring and when I is not valid, then II: the substituents of the B-ring (excluding hydrogens) are not 1-3 methoxy groups, I methoxy group and 1-2 hydroxy groups or 2 methoxy groups and I hydroxy group; or when R₄ and R₅ together form a methylenedioxy group in the A-ring and I and II are not valid, then III: together R₁₃ and R₁₄ R₁₅ and R₁₆ or R₁₇ and R₁₈ is not an oxo group or when R₁₃, R₁₅ and R₁₇ are hydrogens, R₁₄, R₁₆ and R₁₈ is independently not only H, OH or OCH₃; or when R₄ and R₅ together form a methylenedioxy group in the A-ring and I, II and III are not valid, then IV: R₁₃ and R₁₇ or R₁₃ and R₁₅ do not together form a methylenedioxy group, acetonide (dimethyl-methylenedioxy) group or a carbonate (carbonyldioxy) group; or when R₄ and R₅ together form a methylenedioxy group in the A-ring and I, II, III and IV are not valid, then V: the distance between the carbon atom of the methylenedioxy group and the carbon atom of a methoxy group in the B-ring is not 0.85-1.05 nm.

To design an inhibitor of the IGF-1R tyrosine kinase for therapeutic purposes it is of critical importance that the inhibitor does not cross-react with the insulin receptor kinase, which is highly homologous to the IGF-1R. Co-inhibition of the insulin receptor will lead to a diabetogenic response in-vivo. This response comprises a very serious side effect, which cannot be overcome by insulin treatment since the receptor kinase is being blocked. We have recently demonstrated that podophyllotoxin and some congeners are very potent inhibitors of tyrosine phosphorylation of the insulin-like growth factor-I receptor, which plays a pivotal role as a survival factor in cancer cells. Their actions are also highly specific for the IGF-1R, i.e. they do not cross-react with the insulin receptor at all. Moreover, they do not inhibit other major growth factor receptor kinases either. On the other hand podophyllotoxin is a notoriously cytotoxic agent, and although it has been implicated in cancer therapy, severe and unacceptable side effects in patients prevented its use. The anti-cancer effect, as well as the side effects, was attributed to inhibition of microtubule assembly and mitotic block.

The compounds described here are structurally very similar to podophyllotoxin but they can still be sufficiently different (e.g. lack a lactone ring with trans configuration) so that they will be essentially cytotoxic (toxicity is not linked to IGF-1R inhibiton in normal cells).

The invention therefore refers to new and known compounds of the formula I for use as a medicament, and especially for the preparation of a medicament for treatment of IGF-1R dependent diseases, such as cancer, arteriosclerosis, including prevention of restenosis of the coronary arteries after vascular surgery, psoriasis, certain endocrine (e.g. acromegaly) and metabolic disorders (e.g. syndrome X). In addition, the compounds may be used for treatment of virus infected cells and self-reactive lymphocytes (T-cells), when these cells are dependent on IGF-1R for their survival.

The term cancer is used here in a broad sense including carcinomas, i.e. tumours of epithelial origin such as prostatic, breast, gastrointestinal and lung tumours; sarcomas, i.e. mesenchymal tumours such as malignant fibrous histiocytoma and liposarcoma; neuroectodermal tumours such as malignant melanoma, Ewing sarcoma and neuroblastoma; gliomas such as gliobastoma multiforme, astrocytoma and medulloblastoma; myeloproliferative diseases such as myeloma and myeloid leukemia; and lymphoproliferative diseases such as Hodgkin and non-Hodgkin lymphoma and lymphatic leukemia.

In case of tumours not being completely dependent on IGF-1R, the compounds of the invention can be useful to sensitize the tumour cells and potentiate the effect of other anti-cancer treatments. The invention therefore also refers to the use of a compound of the formula I in combination with a cytostaticum or another anti-cancer drug, radiation, radiotherapy, surgery etc. As examples of cytostatica, which can be used together with the compounds of the invention, can be mentioned vincristin, taxol and etoposide.

For parenteral administration, the compounds may be adminis-tered as injectable dosages or by continous intravenous infusion of a solution, suspension or emulsion of the compound in a physiolo-gically acceptable diluent as the pharmaceutical carrier, which can be a sterile liquid, such as water, alcohols, oils, emulsions, and other acceptable organic solvents, with or without the addition of a surfactant and other pharmaceutically acceptable adjuvants.

The compounds can also be administered in the form of a depot injection or implant preparation, which may be formulated in such a manner as to permit a sustained release of the active ingredient.

For oral administration, the compounds can be formulated into solid or liquid preparations such as capsules, pills, tablets, troches, powders, solutions, suspensions or emulsions.

For topical application the compounds can be administered in the form of an unguent, cream, ointment, lotion or a patch.

The results of our biological experiments show that relatively low concentrations of the IGF-1R inhibitors can be sufficient to cause tumour cell death. However, it is believed that it is important to keep a constant plasma concentration of the inhibitors over lengthy periods, to allow them to continuously saturate all IGF-1Rs, and in this way eventually kill as many malignant cells as possible. Therefore, continous infusion of the compounds of the invention, in connection with monitoring the plasma concentration, may be the strategy of treatment instead of repetitive (e.g. daily) injections, which may lead to repeated reactivations of IGF-1R between the treatments.

The invention consequently also refers to a method of treatment of a cancer in a mammal, comprising the steps of administrating a pharmaceutical composition, containing a compound having the formula I in combination with a physiologically acceptable carrier, by constant infusion to a patient suffering from a tumour, monitoring the plasma level of the compound, and adjusting the rate of infusion to keep the plasma level relatively low and relatively constant (depending on the general toxicity of the compound) for a period of time being sufficient for the tumour to be retarded or to disappear.

### EXPERIMENTAL

Materials

Chemicals

Cell culture reagents were purchased from Gibco, Sweden. All other chemicals unless stated otherwise were from Sigma (St. Louis. Mo., USA). A mouse monoclonal antibody against phosphotyrosine (PY99) and a polyclonal antibody against α-subunit of IGF-1R (N20) were obtained from Santa Cruz Biotechnology Inc (Santa Cruz, Calif., USA). A monoclonal antibody against the α-subunit of IGF-1R (IR-3) was purchased from Oncogene Science (N.Y., USA). The murine monoclonal antibody against EGF-R was purchased from Life Science and the Anti-IRS-1 agarose conjugate antibody was obtained from UBI. 4,5-Demethylene-deoxypodophyllotoxin and podophyllotoxin (>99.5% purity) were obtained as gifts from Analytecon SA, Pre Jorat, Switzerland.

Cell Cultures

The human melanoma cell line FM 55 was obtained from Professor R Kiessling, CCK, Karolinska Hospital, Stockholm, Sweden. The P6 cell line, embryonic mouse fibroblasts over-expressing human IGF-1R, was a gift from Professor R. Baserga, Thomas Jefferson University, Philadelphia, Pa., USA.

The cell lines were cultured in Minimal Essential Medium containing 10% fetal bovine serum, glutamine, 1% benzylpenicillin and streptomycin. The cells were grown in monolayers in tissue culture flasks maintained at 95% air/5% CO₂ atmosphere at 37° C. in a humidified incubator. For the experiments cells were cultured in either 35-mm or 60-mm plastic dishes or 96-well plastic plates. The experiments were initiated under subconfluent growth conditions.

Methods

In Vitro Tyrosine Kinase Assays

IGF-1R-catalyzed substrate phosphorylation of polyTyrGlu (pTG) was performed essentially as previously described [Parrizas M., et al., see above, and Blum G., et al., see above]. Immunoprecipitated IR from HepG2, IGF-1R from P6 cell extract and immunodepleted supernatant to assay non-IGF-1R tyrosine kinases. The phosphorylated polymer substrate was probed with a purified phosphotyrosine specific monoclonal antibody conjugated to horseradish peroxidase (HARP). Color was developed with HRP chromogenic substrate o-phenylenediamine dihydrochloride (OPD). The color was quantitated by spectrophotometry (BLISA reader) and reflects the relative amount of tyrosine kinase. The precipitate was immunoblotted with antibodies to IGF-1R and IR to verify the presence of the receptor. Serial dilutions were used to assay the optimal conditions with respect to the amount of IGF-1R and IR. The signal was linear for 30 minutes and was a function of IGF-1R concentration up to 75 ng/well. Briefly, 96 well plates (Immunolon, Nunc) were coated overnight at 4° C. with a mouse monoclonal antibody (LabVision) against the beta-subunit of IGF-1R at a concentration of 1 μg/ml. The plates were blocked with BSA in PBS (ELISA blocking buffer, Pierce), and 80 μg/ml of total protein lysate from the P6 cell line was added. The plates were incubated for 1 h, and washed with PBS Tween. The investigated compounds were added in PBS at room temperature for 30 minutes, prior to kinase activation with IGF-1. Kinase assay was performed using the Sigma kit for in vitro phosphorylation following the manufacturer instructions.

IGF-1R tyrosine autophosphorylation was analysed by a sandwich ELISA assay. Briefly, 96-well plates (Immunolon, Nunc) were coated overnight at 4° C. with 1 μg/well of the monoclonal antibody Ab-5 (LabVision) to the IGF-1R beta subunit. The plates were blocked with 1% BSA in PBS Tween for 1 h, then 80 g/well of total protein lysate from the P6 cell line was added. As a negative control was used total protein lysate from R-cell line. The investigated compounds were added in tyrosine kinase buffer without ATP at room temperature for 30 min, prior to kinase activation with ATP. Kinase assay was performed using the Sigma kit.

Assay of Tyrosine Phosphorylation of Receptors in Intact Cells

Cells were cultured to subconfluency in 6-cm plates, and then fresh medium containing 10% FBS and the desired compounds were added for 1 h. The cells were then lyzed and subjected to immunoprecipitation using specific antibodies. Immunoprecipitates were resolved by sodium dodecyl sulphate polyacrylamide gel electrophoresis (SDS-PAGE) and transferred to nitro-cellulose membranes and incubated with anti-phosphotyrosine antibody. Antibodies to actin (in cell extract) or IGF-1R beta subunit were used as loading controls. After detection the films were scanned for quantification.

Immunoprecipitation and Determination of Protein Content

The isolated cells were then lyzed in 10 ml ice-cold PBSTDS containing protease inhibitors (Carlberg, M., et al., J Biol Chem 271:17453-17462, 1996). 50 μl protein A or G agarose was added in 1 ml sample and incubated for 15 min at 4° C. on an orbital shaker. After centrifugation for 10 min at 10,000 r/min at 4° C. the supernatant was saved. The protein content was determined by a dye-binding assay with a reagent purchased from Bio-Rad. Bovine serum albumin was used as a standard. 15 μl Protein G Plus agarose and 5 μl anti-IGF-1R were added. After a 3 h incubation at 4° C. on an orbital shaker the precipitate was collected by pulse centrifugation in a micro centrifuge at 14,000×g for 10 s. The supernatant was discarded and the pellet was washed 3 times with PBSTDS.

Sodium Dodecyl Sulphate Polyacrylamide Gel Electrophoresis (SDS-PAGE)

Protein samples were solved in a 2×-sample buffer containing Laemmli buffer and 0.5% methanol and boiled for 5 min at 96° C. Samples were separated by SDS-PAGE with a 4% stacking gel and 7.5% separation gel. Molecular weight markers (Bio Rad, Sweden) were run simultaneously in all experiments.

Western Blotting

Following SDS-PAGE the proteins were transferred overnight to nitro-cellulose membranes (Hybond, Amersham, UK) and then blocked for 1 h at room temperature in a solution of 4% skimmed milk powder and 0.02% Tween 20 in PBS, pH 7.5. Incubations with the primary antibodies were performed for 1 h at room temperature, followed by 3 washes with PBS with Tween and incubation with the second antibody for 1 h room temperature. After another 3 washes the membranes were incubated with Streptavidin-labelled horseradish peroxidase for 30 min and then detected using Amersham ECL system (Amersham, UK). The films were scanned by Fluor-S (BioRad).

### Experiment 1

Effect of 4,5-demethylene-deoxypodophyllotoxin and podophyllotoxin on phosphorylation of IGF-1R in Cultured Melanoma Cells

Melanoma cells (line FM55) were seeded in 6-cm dishes, at a concentration of 10,000 cells/cm2 in Minimal Essential Medium supplemented with 10% fetal calf serum (FCS). When the cells reached a density of 65,000 cells/cm² in the dishes, they were treated for 1 h with 4,5-demethylene-deoxypodophyllotoxin (0.7 μM) and podophyllotoxin (used as a positive control; 0.7 μM). Treatment with 0 μM represents untreated controls. The cells were then harvested and subjected to immunoprecipitation of the IGF-1R. The immunoprecipitates, containing purified IGF-1R, were fractionated by gel electrophoresis. Phosphorylation of IGF-1R was detected by an anti-phosphotyrosine antibody using Western blotting. The obtained signals represent phosphorylated IGF-1R and the intensity of signals represents amounts of phosphorylated IGF-1R. Details of the methods used are described above. The intensities are quantified by a scanner, which measures the optical density (OD) of the signals. For the control cells the OD is set at 100%. The blank (OD 0%) represents the background.

TABLE 1
Level of IGF-1R phosphorylation in intact cells (% OD)

| | |
|---|---|
| Podophyllotoxin | 10 |
| 4,5-demethylene-deoxypodophyllotoxin | 73 |

The results show that 4,5-demethylene-deoxypodophyllotoxin can inhibit of IGF-1R phosphorylation, although being less potent than podophyllotoxin.

### Experiment 2

Effect of 4,5-demethylene-deoxypodophyllotoxin on phosphorylation of IGF-1R in a Cell-Free System

We isolated the receptor and determined the effects of 4,5-demethylene-deoxypodophyllotoxin on IGF-1R catalyzed substrate tyrosine phosphorylation and IGF-1R autophosphorylation in-vitro. 4,5-Demethylene-deoxypodophyllotoxin significantly (by about 35% at a concentration of 0.5 μM) decreased the pTG substrate phosphorylation by the IGF-1 receptor. In contrast, it failed to interfere with substrate phosphorylation of epidermal growth factor receptor and insulin receptor tyrosine kinases, as well as that of other ‘non-IGF-1R kinases’, which were obtained by immunodepletion of IGF-1R (data not shown). Podophyllotoxin, used here as a positive control, produced similar result, i.e. had only effect on IGF-1R. In the next set of experiments we found that both podophyllotoxin and 4,5-demethylene-deoxypodophyllotoxin inhibited autophosphorylation of IGF-1R in vitro. A stronger response was obtained by podophyllotoxin.

Taken together, these data imply that both podophyllotoxin and 4,5-demethylene-deoxypodophyllotoxin inhibit the IGF-1R tyrosine kinase.

### Experiment 3

Specificity of 4,5-demethylene-deoxypodophyllotoxin and podophyllotoxin on Various Receptor Tyrosine Kinases in Cultured Cells

FM55 melanoma cells were cultured in the same way as described in Experiment 1. When reaching a density of 65,000 cells/cm² in the dishes, they were treated for 1 h with 0 (control) and of 4,5-demethylene-deoxypodophyllotoxin (0.7 μM) and podophyllotoxin (positive control; 0.7 μM). The cells were then isolated and subjected to immunoprecipitation of the IGF-1R, fibroblast growth factor receptor (FGFR), platelet-derived growth factor receptor (PDGFR), epidermal growth factor receptor (EGFR), insulin receptor (IR) and insulin substrate-1 (IRS-1) using antibodies to respective molecules. IRS-1 is a substrate of IGF-1R, and therefore its phosporylation is dependent on phosphorylated IGF-1R. The results are shown in Table 2.

Gelelectrophoresis, Western blotting and quantification of the different signals were performed as described above.

TABLE 2
Level of IGF-1R phosporylation in intact cells (% OD)

Substance

| | 4,5-DM- | |
|---|---|---|
| Substrate | deoxypodophyllotoxin | Podophyllotoxin |
| IGF-1R | 65 | 12 |
| FGFR | 104 | 99 |
| PDGFR | 99 | 101 |
| EGFR | 100 | 102 |
| IR | 102 | 103 |
| IRS-1 | 70 | 9 |

This demonstrates that 4,5-demethylene-deoxypodophyllotoxin and podophyllotoxin are specific for IGF-1R.

### CONCLUSION.

It has been demonstrated that 4,5-demethylene-deoxypodophyllotoxin, like podophyllotoxin, is a highly specific inhibitor of the insulin-like growth factor-1 receptor (IGF-1R) tyrosine kinase. This finding shows that there are additional derivatives of cyclolignans and related compounds, which can selectively inhibit the IGF-1R activity in cells, thereby increasing the chance of finding non-toxic and pharmacologically suitable inhibitors. This new mechanism of action of the described compounds will be useful when developing new theraputic regimen for cancer and other IGF-1R dependent diseases.

1. Use of a compound having the formula I 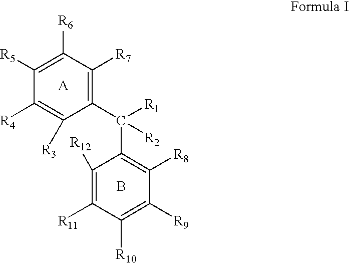
wherein A and B are rings linked together by a bridge C and independently represent benzene, pyridine or pyrimidine rings;
the bridge C is a carbon atom, a nitrogen, an oxygen or a sulphur atom;
R₁ and R₂ independently are H, OH, a C₁₋₆ linear or branched alkoxy chain or a C₁₋₂₀ linear or branched alkyl chain, or a C₁₋₆ linear or branched alkoxy or C₁₋₂₀ linear or branched alkyl chain having one or more of 1-2 double bonds, 1 triple bond, 1-4 oxygen functions, 1-3 nitrogen-, 1-3 halogen- or 1-2 sulphur-containing substituents, 1-2 phosphate groups, 1-2 non-substituted or substituted phenyl or cyclohexyl groups, or 1-2 five- or six-membered heterocyclic rings; or R₁ and
R₇ together are said alkyl or alkoxy chain bonded directly to a carbon atom in the ring A or via a carbon, oxygen, nitrogen or sulphur atom; or R₁ and R₂ together form a double bond to the optionally substituted C₁₋₂₀ linear or branched allyl chain, to an oxo group, to a sulphur atom or to a nitrogen atom substituted with H, OH, an alkyl or alkoxy group; and
R₃, R₄, R₅ R₆, R₇, R₈, R₉, R₁₀, R₁₁ and R₁₂, which can be the same or different, are selected from the group consisting of H, OH, OCH₃, OCH₂CH₃, OCH₂CH₂CH₃, OCH(CH₃)₂, OC(CH₃)₃, OCHCH₂, OCHCHCH₃, OCH₂CHCH₂, OCCH, OCOH, OCO(CH₂)₀₋₁₈CH₃, OCH₂OH, OCHO, OCOOH, OCOCH₃, OCOC₂H₅, OCOC₃H₇, OCOOCH₃, OCOOC₂H₅, OCOOC₃H₇, OCH₂OOCH, OCH₂OOCCH₃, OCH₂OOCC₂H₅, OCH₂CH₂OH, OCH₂CHO, OCH₂COOH, OC₂H₄CH₂OH, OC₂H₄CHO, OC₂H₄COOH, CH₃, CH₂CH₃, CH₂CH₂CH₃, CH(CH₃)₂, C(CH₃)₃, CHCH₂, CHCHCH₃, CH₂CHCH₂, CCH, CH₂OH, CHO, COOH, COCH₃, COC₂H₅, COC₃H₇, COOCH₃, COOC₂H₅, COOC₃H₇, CH₂OOCH, CH₂OOCCH₃, CH₂OOCC₂H₅, CH₂CH₂OH, CH₂CHO, CH₂COOH, C₂H₄CH₂OH, C₂H₄CHO, C₂H₄COOH, F, Cl, Br, I, CF₃, CN, NH₂, NO₂, CH₂CN, CH₂NH₂, CH₂NO₂, CONH₂, CONHCH₃, NH(C₁₋₃ alkyl), N(C₁₋₃ alkyl)₂, NHCOCH₃, NHCOCH₃, NHNHCONH₂, SCH₃, OPO₃ and OSi(CH₃)₂C(CH₃)₃; or
wherein two of R₃, R₄, R₅, R₆, R₇, R₈, R₉, R₁₀, R₁₁ and R₁₂ on adjacent carbon atoms in the rings A and B together are CH₂CH₂CH₂, CH₂CH₂CH₂CH₂, O(CH₂)₁₋₃O, OCHCH₃O, OC(CH₃)₂O, OCOO, OCOCH₂, NHCH₂CH₂, or NHCOCH₂;
with the provisos that when R₄ and R₅ together form a methylenedioxy group:
i) R₃ and R₆ must not both be H when R₇ is H or forms a bond with R₁ being a C₁₋₅ linear or branched alkyl chain optionally having a double bond; or
ii) R₈, R₉, R₁₀, R₁₁ and R₁₂ must not be 1-3 OCH₃ and 2-4 H, or 1 OCH₃ and 2 OH and 2 H, or 1 OCH₃ and 1 OH and 3 H, or 2 OCH₃ and 1 OH and 2 H; or
iii) and when R₂ is H, then R₁ must not be H, OH, OCH₃, OC₂H₅, or a C₁₋₅ linear or branched alkyl chain optionally having a double bond or together with R₇ forming a bond to the carbon atom in the ring A, and with 0-3 oxygen functions; or
iv) and when R₂ is H, then the distance between the carbon atom of the methylenedioxy group and the carbon atom of a methoxy group in the ring B must not be 0.85-1.05 nm;
as a specific inhibitor of tyrosine phosphorylation of the insulin-like growth factor-1 receptor.

2. Use according to claim 1 of a compound having the formula Ib 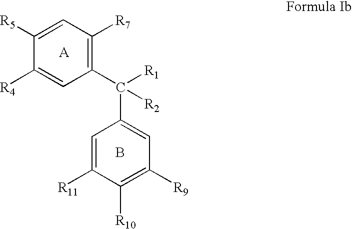
wherein A and B represent benzene rings and C is a carbon atom;
R₁ and R₂, which can be the same or different, are selected from the group consisting of H, OH, CH₃, CH₂CH₃, CH₂CH₂CH₃, CH₂CH₂CH₂CH₃, CH₂CHCH₂, CH₂CH(CH₃)₂, OCH₃, OCH₂CH₃, OCH₂CH₂CH₃, OCH₂CH₂CH₂CH₃, OCH₂CHCH₂, OCH₂CH(CH₃)₂, CH₂OH, CH₂CH₂OH, CH₂CH₂CH₂OH, OCH₂OH, OCH₂CH₂OH, OCH₂CH₂CH₂OH, a phenyl, piperidinyl and morpholino group; or R₁ and R₂ together are O, CH₂, CHCH₃, CHCH₂CH₃, C(CH₃)₂, CHCH(CH₃)₂, C(CH₂CH₃)phenyl, NOH, NOCH₃, NOCH₂CH₃ or NOCH₂CH₂CH₃; and
R₄, R₅, R₇, R₉, R₁₀ and R₁₁, which may be the same or different, are selected from the group consisting of H, OH, CH₃, CH₂CH₃, CH₂CH₂CH₃, CH₂CHCH₂, OCH₃, OCH₂CH₃, OCH₂CH₂CH₃, OCH₂CHCH₂, CH₂OH, CH₂CH₂OH, OCH₂OH, OCH₂CH₂OH, COOCH₃, F, Cl, CF₃, NH₂, NHCH₃, OCO(CH₂)₀₋₁₈CH₃ and OPO₀₃; or wherein R₄ and R₅ and/or R₉ and R₁₀ together are a methylenedioxy group;
with the provisos that when R₄ and R₅ together form a methylenedioxy group:
i) R₇ must not be H; or
ii) R₉, R₁₀, and R₁₁ must not be 1-3 OCH₃ and 0-2 H, or 1 OCH₃ and 2 OH, or 1 OCH₃ and 1 OH and 1 H, or 2 OCH₃ and 1 OH; or
iii) and when R₂ is H, then R₁ must not be H, OH, OCH₃, OC₂H₅, or a C₁₋₅ linear or branched alkyl chain optionally having a double bond and/or 1-3 oxygen functions; or
iv) and when R₂ is H, then the distance between the carbon atom of the methylenedioxy group and the carbon atom of a methoxy group in the ring B must not be 0.85-1.05 nm.

3. Use according to claim 1 of a compound of the formula II 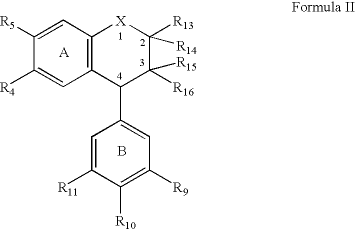
wherein A and B represent benzene rings;
X is O, NH, NCH₃, NCH₂CH₃, NOH, NOCH₃, S or SO₂;
R₄, R₅, R₉, R₁₀ and R₁₁ , which may be the same or different, are selected from the group consisting of H, OH, CH₃, CH₂CH₃, CH₂CH₂CH₃, CH₂CHCH₂, OCH₃, OCH₂CH₃, OCH₂CH₂CH₃, OCH₂CHCH₂, CH₂OH, CH₂CH₂OH, OCH₂OH, OCH₂CH₂OH, COOCH₃, F, Cl, CF₃, NH₂, NHCH₃, OCO(CH₂)₀₋₁₈CH₃ and OPO₃; or R₄ and R₅ and/or R₉ and R₁₀ together are a methylenedioxy group; and
R₁₃, R₁₄, R₁₅ and R₁₆, which can be the same or different, are selected from the group consisting of H, OH, CH₃, CH₂CH₃, CH₂CH₂CH₃, CH₂CH₂CH₂CH₃, CH₂CHCH₂, CH₂CH(CH₃)₂, OCH₃, OCH₂CH₃, OCH₂CH₂CH₃, OCH₂CH₂CH₂CH₃, OCH₂CHCH₂, OCH₂CH(CH₃)₂, CH₂OH, CH₂CH₂OH, CH₂CH₂CH₂OH, OCH₂OH, OCH₂CH₂OH, OCH₂CH₂CH₂OH, a phenyl, piperidinyl and morpholino group; or R₁₄ and R₁₅
together form a double bond between the carbon atoms 2 and 3.

4. Use of a compound of the formula II according to claim 3, wherein R₁₃ and R₁₄ together or R₁₅ and R₁₆ together are O, or R₁₄ and R₁₅ together are selected from the group consisting of CH₂OCO, COOCH₂, CH₂OCH₂, CH₂CH₂CO, CH₂OC(CH₃)₂OCH₂, OC(CH₃)₂O, CH₂OCOOCH₂, OCOO, CH₂OCH₂OCH₂ and OCH₂O.

5. Use according to claim 1 of a compound of the formula III 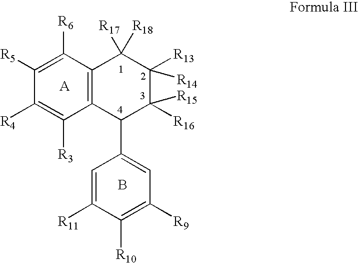
wherein A and B represent benzene rings;
R₃, R₄, R₅, R₆, R₉, R₁₀ and R₁₁, which may be the same or different, are selected from the group consisting of H, OH, CH₃, CH₂CH₃, CH₂CH₂CH₃, CH₂CHCH₂, OCH₃, OCH₂CH₃, OCH₂CH₂CH₃, OCH₂CHCH₂, CH₂OH, CH₂CH₂OH, OCH₂OH, OCH₂CH₂OH, COOCH₃, F, Cl, CF₃, NH₂, NHCH₃, OCO(CH₂)₀₋₁₈CH₃ and OPO₃; or R₄ and R₅ and/or R₉ and R₁₀ together are a methylenedioxy group;
R₁₃, R₁₄, R₁₅, ₁₆, ₁₇ and R₁₈, which can be the same or different, are selected from the group consisting of H, OH, OCH₃, OCH₂CH₃, OCH₂CH₂CH₃, OCH₂CH₂CH₂CH₃, OCH₂CHCH₂, OCH₂CH(CH₃)₂, CH₂OH, CH₂CH₂OH, CH₂CH₂CH₂OH, OCH_{2 OH, OCH}₂CH₂OH, OCH₂CH₂CH₂OH, a phenyl, piperidinyl and morpbolino group; or R₁₅, R₁₆, R₁₇ and R₁₈ are CH₃, CH₂CH₃, CH₂CH₂CH₃, CH₂CH₂CH₂CH₃, CH₂CHCH₂, or CH₂CH(CH₃)₂; or R₁₃ and R₁₄ together or
R₁₄ and R₁₅ together or R₁₇ and R₁₈ together are O; or R₁₄ and R₁₅ together are OC(CH₃)₂O, OCOO or OCH₂O; or R₁₄ and R₁₅ form a double bond between the carbon atoms 2 and 3; with the provisos that when R₄ and R₅ together form a methylenedioxy group:
i) R₃ and R₆ must not both be H; or
ii) R₉, R₁₀ and R₁₁, must not be 1-3 OCH₃ and 0-2 H, or 1 OCH₃ and 2 OH, or 1 OCH₃ and 1 OH and 1 H, or 2 OCH₃ and 1 OH; or
iii) R₁₃ and R₁₄ together, R₁₅ and R₁₆ together, or R₁₇ and R₁₈ together must not be O; or
iv) and when R₁₃, R₁₅ and R₁₇ are H, R₁₄, R₁₆ and R₁₈ must not be only H, OH or OCH₃ or R₁₄ and R₁₆ together or R₁₄ and R₁₈ together must not form a methylenedioxy group, acetonide group or a carbonate group; or
v) the distance between the carbon atom of the methylenedioxy group and the carbon atom of a methoxy group in the ring B must not be 0.85-1.05 nm.

6. Use according to claim 1 of a compound of the formula IV 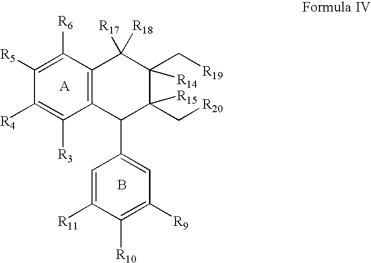
wherein A and B represent benzene rings;
R₃, R₄, R₅, R₆, R₉, R₁₀ and R₁₁, which may be the same or different, are selected from the group consisting of H, OH, CH₃, CH₂CH₃, CH₂CH₂CH₃, CH₂CHCH₂, OCH₃, OCH₂CH₃, OCH₂CH₂CH₃, OCH₂CHCH₂, CH₂OH, CH₂CH₂OH, OCH₂OH, OCH₂CH₂OH, COOCH₃, F, Cl, CF₃, NH₂, NHCH₃, OCO(CH₂)₀₋₁₈CH₃ and OPO₃; or R₄ and R₅ and/or R₉ and R₁₀ together are a methylenedioxy group;
R₁₄ and R₁₅, which can be the same or different, are H, OH, CH₃ or OCH₃;
R₁₇ and R₁₈, which can be the same or different, are H, OH, CH₃,CH₂CH₃,OCOH, OCO(CH₂)₀₋₁₈CH₃, OCH₃, OC₂H₅ or OPO3; or R₁₇ and R₁₈ together are O, CH₂, CHCH₃, NOH, NOCH₃, NOCH₂CH₃;
R₁₉ and R₂₀, which can be the same or different, are H, OH, OCH₃, OC₂H₅ OOCH₃, OOCH₂CH₃, OCOH, or OCO(CH₂)₀₋₁₈CH₃; or R₁₉ and R₂₀ together are O, CH₂, CHCH₃, NOH, NOCH₃, NOCH₂CH₃ or a methylene bridge, an ether or a lactone group;
with the provisos that when R₄ and R₅ together form a methylenedioxy group:
i) R₃ and R₆ must not both be H; or
ii) R₉, R₁₀ and R₁₁ must not be 1-3 OCH₃ and 0-2 H, or 1 OCH₃ and 2 OH, or 1 OCH₃ and 1 OH and 1 H, or 2 OCH₃ and 1 OH; or
iii) and when R₁₄, R₁₅ and R₁₇ are H, then R₁₈, R₁₉ and R₂₀ must not only be H. OH, OCH₃ or OC₂H₅, or R₁₉ and R₂₀ must not be OOCH₃ or OOCH₂CH₃ or R₁₉ and R₂₀ together must not be an ether or a lactone group; or
iv) and when R₁₄ and R₁₅ are H, then R₁₇ and R₁₈ together must not be O in combination with R₁₉ and R₂₀ being H, OH, OCH₃ or OC₂H₅, or R₁₉ and R₂₀ being OOCH₃ or OOCH₂CH₃ or R₁₉ and R₂₀ together being an ether or a lactone group; or
v) the distance between the carbon atom of the methylenedioxy group and the carbon atom of a methoxy group in the ring B must not be 0.85-1.05 nm.

7. Use according to claim 6 of a compound selected from the group consisting of 4,5-demethylene-deoxypodophyllotoxin, 4,5-dimethoxy-deoxypodophyllotoxin, 4,5-dimethoxy-deoxypicropodophyllin, beta-picropeltatin, beta-picropeltatin disodium phosphate, beta-picropeltatin valerate, picropodophyllin disodium phosphate, picropodophyllin valerate, austrobailignan 1, austrobailignan 2, austrobailignan 3, polygamatin and picropolygamatin.

8. A compound of the formula III 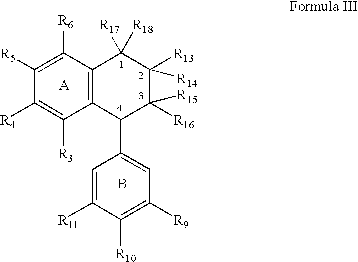
wherein A and B represent benzene rings;
₃, R₄, R₅, R₆, R₉, R₁₀ and R ₁₁, which can be the same or different, are selected from the group consisting of H, OH, CH₃, CH₂CH₃, CH₂CH₂CH₃, CH₂CHCH₂, OCH₃, OCH₂CH₃, OCH₂CH₂CH₃, OCH₂CHCH₂, CH₂OH, CH₂CH₂OH, OCH₂OH, OCH₂CH₂OH, COOCH₃, F, Cl, CF₃, NH₂, NHCH₃, OCO(CH₂)₀₋₁₈CH₃ and OPO₃; or R₄ and R₅ and/or R₉ and R₁₀ together are a methylenedioxy group;
R₁₃, R₁₄, R₁₅, R₁₆, R₁₇ and R₁₈, which can be the same or different, are selected from the group consisting of H, OH, OCH₃, OCH₂CH₃, OCH₂CH₂CH₃, OCH₂CH₂CH₂CH₃, OCH₂CHCH₂, OCH₂CH(CH₃)₂, CH₂OH, CH₂CH₂OH, CH₂CH₂CH₂OH, OCH₂OH, OCH₂CH₂OH, OCH₂CH₂CH₂OH, a phenyl, piperidinyl and morpholino group; or R₁₅, R₁₆, R₁₇ and R₁₈ are CH₃, CH₂CH₃, CH₂CH₂CH₃, CH₂CH₂CH₂CH₃,CH₂CHCH₂, or CH₂CH(CH₃)₂; or R₁₃ and R₁₄ together or R₁₄ and R₁₅ together or R₁₇ and R₁₈ together are O; or R₁₄ and R₁₅ together are OC(CH₃)₂O, OCOO or OCH₂O; or R₁₄ and R₁₅ form a double bond between the carbon atoms 2 and 3;
with the provisos that when R₄ and R₅ together form a methylenedioxy group:
i) R₃ and R₆ must not both be H; or
ii) R₉, R₁₀ and R₁₀ must not be 1-3 OCH₃ and 0-2 H, or 1 OCH₃ and 2 OH, or 1 OCH₃ and 1 OH and 1 H, or 2 OCH₃ and 1 OH; or
iii) R₁₃ and R₁₄ together, R₁₅ and R₁₆ together, or R₁₇ and R₁₈ together must not be O; or
iv) and when R₁₃, R₁₅ and R₁₇ are H, R₁₄, R₁₆ and R₁₈ must not be only H, OH or OCH₃ or R₁₄ and R₁₆ together or R₁₄ and R₁₈ together must not form a methylenedioxy group, acetonide group or a carbonate group; or
v) the distance between the carbon atom of the methylenedioxy group and the carbon atom of a methoxy group in the ring B must not be 0.85-1.05 nm.

9. A compound according to claim 8 for use as a medicament.

10. A pharmaceutical composition comprising a compound of the formula I 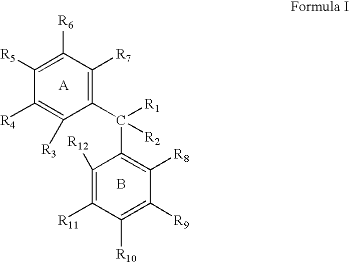
wherein A and B are rings linked together by a bridge C and independently represent benzene, pyridine or pyrimidine rings;
the bridge C is a carbon atom, a nitrogen, an oxygen or a sulphur atom;
R₁ and R₂ independently are H, OH, a C₁₋₆ linear or branched alkoxy chain or a C₁₋₂₀ linear or branched alkyl chain, or a C₁₋₆ linear or branched alkoxy or C₁₋₂₀ linear or branched alkyl chain having one or more of 1-2 double bonds, 1 triple bond, 1-4 oxygen fuictions, 1-3 nitrogen-, 1-3 halogen- or 1-2 sulphur-containing substituents, 1-2 phosphate groups, 1-2 non-substituted or substituted phenyl or cyclohexyl groups, or 1-2 five- or six-membered heterocyclic rings; or R₁ and R₇ together are said alkyl or alkoxy chain bonded directly to a carbon atom in the ring A or via a carbon, oxygen, nitrogen or sulphur atom; or R₁ and R₂ together form a double bond to the optionally substituted C₁₋₂₀ linear or branched alkyl chain, to an oxo group, to a sulphur atom or to a nitrogen atom substituted with H. OH, an alkyl or alkoxy group; and
R₃, R₄, R₅, R₆, R₇, R₈, R₉, R₁₀, R₁₁ and R₁₂, which can be the same or different, are selected from the group consisting of H, OH, OCH₃, OCH₂CH₃, OCH₂CH₂CH₃, OCH(CH₃)₂, OC(CH₃)₃, OCHCH₂, OCHCHCH₃, OCH₂CHCH₂, OCCH, OCOH, OCO(CH₂)₀₋₁₈CH₃, OCH₂OH, OCHO, OCOOH, OCOCH₃, OCOC₂H₅, OCOC₃H₇, OCOOCH₃, OCOOC₂H₅, OCOOC₃H₇, OCH₂OOCH OCH₂OOCCH₃, OCH₂OOCC₂H₅, OCH₂CH₂OH, OCH₂CHO, OCH₂COOH, OC₂H₄CH₂OH, OC₂H₄CHO, OC₂H₄COOH, CH₃, CH₂CH₃, CH₂CH₂CH₃, CH(CH₃)₂, C(CH₃)₃, CHCH₂, CHCHCH₃, CH₂CHCH₂, CCH, CH₂OH, CHO, COOH, COCH₃, COC₂H₅, COC₃H₇, COOCH₃, COOC₂H₅, COOC₃H₇, CH₂OOCH, CH₂OOCCH₃, CH₂OOCC₂H₅, CH₂CH₂OH, CH₂CHO, CH₂COOH, C₂H₄CH₂OH, C₂H₄CHO, C₂H₄COOH, F, Cl, Br, I, CF₃, CN, NH₂, NO₂, CH₂CN, CH₂NH₂, CH₂NO₂, CONH₂, CONHCH₃, NH(C₁₋₃ alkyl), N(C₁₋₃ alkyl)₂, NHCOCH₃, NHNHCOCH₃, NHNHCONH₂, SCH₃, OPO₃ and OSi(CH₃)₂C(CH₃)₃; or
wherein two of R₃, R₄, R₅, R₆, R₇, R₈, R₉, R₁₀, R₁₁ and R₁₂ on adjacent carbon atoms in the rings A and B together are CH₂CH₂CH₂, CH₂CH₂CH₂CH₂, O(CH₂)₁₋₃O, OCHCH₃O, OC(CH₃)₂O, OCOO, OCOCH₂, NHCH₂CH₂, or NHCOCH₂;.
with the provisos that when R₄ and R₅.together form a methylenedioxy group:
i) R₃ and R₆ must not both be H when R₇ is H or forms a bond with R₁ being a C₁₋₅ linear or branched alkyl chain optionally having a double bond; or
ii) R₈, R₉, R₁₀, R₁₁ and R₁₂ must not be 1-3 OCH₃ and 2-4 H, or 1 OCH₃and 2 OH and 2 H, or 1 OCH₃ and 1 OH and 3 H, or 2 OCH₃ and 1 OH and 2 H; or
iii) and when R₂ is H, then R₁ must not be H, OH, OCH₃, OC₂H₅, or a C₁₋₅ linear or branched alkyl chain optionally having a double bond or together with R₇ forming a bond to the carbon atom in the ring A, and with 0-3 oxygen functions; or
iv) and when R₂ is H, then the distance between the carbon atom of the methylenedioxy group and the carbon atom of a methoxy group in the ring B must not be 0.85-1.05 nm;
in combination with a physiologically acceptable carrier.

11. A pharmaceutical composition according to claim 10, comprising a compound having the formula I, Ib, II, III or IV as defined in any of claims 2-8.

12. Use of a compound having the formula I, Ib, II, III or IV as defined in any of claims 1-8 for the preparation of a medicament specifically inhibiting tyrosine phosphorylation of the insulin-like growth factor-i receptor.

13. Use of a compound having the formula I, Ib, II, III or IV as defined in any of claims 1-8 for the preparation of a medicament for prophylaxis or treatment of IGF-1R dependent diseases in vertebrates, such as benign and malignant neoplasms, the latter including carcinomas, sarcomas, neuroectodermal tumours, gliomas, myeloproliferative and lymphoproliferative diseases, and arteriosclerosis, restenosis of the coronary arteries after vascular surgery, psoriasis, certain endocrine disorders, such as acromegaly, and metabolic disorders, such as syndrome X, and also for treatment of virus infected cells and self-reactive lymphocytes (T-cells), when these cells are dependent on IGF-1R for their survival.

14. Use according to claim 13 for prophylaxis or treatment in mammals, especially humans.

15. Use of a compound having the formula I, Ib, II, III or IV as defined in any of claims 1-8 in combination with other anti-cancer treatments, including cytostatica and other anti-cancer drugs, radiation, radiotherapy and surgery, for treatment of cancer in mammals.

16. Method of treatment of a cancer in a mammal, comprising the steps of administrating a pharmaceutical composition according to claim 10 or 11 by constant infusion to a patient suffering from a tumour, monitoring the plasma level of the compound, and adjusting the rate of infusion to keep the plasma level relatively low and relatively constant for a period of time being sufficient for the tumour to be retarded or to disappear.
